(19) United States
(12) Patent Application Publication
(10) Pub. No.: US 2007/0099986 A1
(43) Pub. Date: May 3, 2007

(54) Preventives/remedies for urinary disturbance

(76) Inventors: Yuji Ishichi, Osaka (JP); Koichi Iwanaga, Osaka (JP); Tomomi Ikemoto, Osaka (JP); Hiroaki Yamamoto, Osaka (JP); Shokyo Miki, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 11/589,903

(22) Filed: Oct. 31, 2006

(30) Foreign Application Priority Data
Nov. 2, 2005 (JP) 2005-319789

(51) Int. Cl. A61K 31/343; A61K 31/36; C07D 319/14; A61K 31/18; C07C 311/18

(52) U.S. Cl. 514/452; 514/469; 514/602; 564/86; 549/467; 549/362

(57) ABSTRACT

A compound represented by the formula: 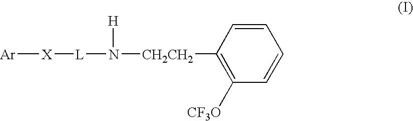
wherein Ar represents a group represented by the formula: 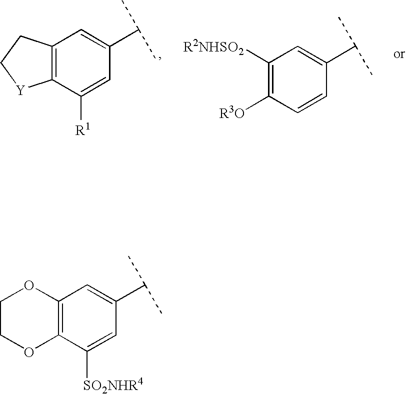
(wherein Y represents methylene or an oxygen atom, R¹ represents aminosulfonyl, C₁₋₆ alkylaminosulfonyl, C₁₋₆ alkylcarbonylamino or C₁₋₆ alkylsulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group; and L represents an optionally substituted C₄₋₅ alkylene group, or a salt thereof is provided.

### TECHNICAL FIELD

The present invention relates to pharmaceutical compositions, in particular to an agent for preventing and/or treating lower urinary tract symptoms.

### BACKGROUND ART

Lower urinary tract symptoms are composed of the subjective and the objective ones in a process from accumulation of urine (urinary storage) through excretion (urinary voiding), and they are classified into urinary storage symptoms (urinary incontinence, urinary frequency, etc.), voiding symptoms (voiding difficulty, pain on micturition, obstruction of urinary tract, etc.), and the like. Lower urinary tract symptoms in the elderly, particularly voiding difficulty, especially voiding difficulty caused by benign prostatic hyperplasia, is a great social problem with the advance of a recent aging society, though these symptoms are also found in the youth.

Urinary voiding (micturition) is, under the control of the micturition centers, controlled by the peripheral nervous systems involving the parasympathetic nerve such as pelvic nerve, the sympathetic nerve such as hypogastric nerve, and somatic nerves such as pudendal nerve and nerves to pelvic floor muscles, and it is suggested that a variety of neurotransmitters (e.g., acetylcholine, noradrenaline, ATP, substance P, neuropeptide Y, etc.) are involved in the voiding cycles.

As an agent for treating lower urinary tract symptoms, particularly voiding difficulty, those for increasing contraction of muscle of urinary bladder (detrusor) or those for relaxing sphincter smooth muscle of urethra to reduce urethral resistance have been used. The agents acting on the detrusor muscle to increase the contraction, for example, cholinergic agents such as bethanechol, acetylcholinesterase inhibitors such as distigmine, and the like have been used. However, for example, bethanechol is incompatible with pregnant women, peptic ulcers, organic ileus, asthma, hyperthyroidism, etc., because it contracts the detrusor muscle in the urinary storage phase to decrease the bladder capacity, while exhibiting side-effects such as epiphora, sweating, gastro-intestinal disorders, and stomachache. No satisfied drug is available clinically.

As the acetylcholinesterase inhibitors increasing the contraction of detrusor muscle, distigmine, neostigmine, etc. are known. The acetylcholinesterase inhibitors increase the bladder contractility during voiding by enhancing the effect of acetylcholine released from the peripheral end of the pelvic nerves, and thus they are considered as drugs excellent in physiological systems of voiding. However, it is known that, for example, while distigmine increases the bladder contractility, it also causes the urethral smooth muscle contractions by the direct activation of nicotinic receptors to increase urethral resistance during voiding. Then, the clinical effects of this drug are insufficient with the low voiding efficiency and this agent has a risk of causing the high pressure voiding. In addition, neostigmine has not been used for the clinical treatment because of the short duration of the action (see, for example, Takamichi Hattori and Kosaku Yasuda, “Sinkeiinseiboukou-No-Sindan-To-Chiryou (Diagnosis and Therapy of Neurogenic Bladder)”, 2nd Ed., p. 105-106, p. 139, Igaku-Shoin Ltd., Tokyo.).

As a drug which relaxes the urethral smooth muscle to decrease the urethral resistance, for example, an α₁ receptor antagonist such as Tamusulosin, Prazosin, Alfuzosin, Naftopidil, and Urapidil are used. These are reported to be effective in the amelioration of symptoms such as feeling of incomplete emptying and nocturia, but they cause adverse effects such as orthostatic hypotension, etc., thus careful observations are required.

EP 1118322 A describes an acetylcholinesterase inhibitor, an agent for preventing and/or treating lower urinary tract symptoms (especially voiding difficulty), and it reports that a combined use of an α₁ receptor antagonist and an acetylcholinesterase inhibitor improves the flow rate of urine.

Further, EP 1466625 A describes a compound having a combined effect of an acetylcholinesterase inhibitory action and an α₁ receptor antagonistic action, and a compound represented by the formula: 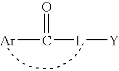
wherein Ar represents a 5- or 6-membered aromatic ring group which may be condensed, whose aromatic ring is optionally substituted, L represents a spacer having a main chain of 1 to 10 atoms, or may be taken with Ar to form a ring, and Y represents an optionally substituted amino group or an optionally substituted nitrogen-containing heterocyclic group, as an agent for preventing and/or treating lower urinary tract symptoms (especially voiding difficulty).

However, there is no specific description of the presently claimed compounds of the present invention.

### DISCLOSURE OF THE INVENTION

Therefore, it is an object of the present invention to develop an agent for preventing and/or treating lower urinary tract symptoms, particularly voiding difficulty, which has higher effectiveness to thess symptoms, higher convenience and less side-effects, as compared with known compounds or a combination thereof.

Under these circumstances, the present inventors have conducted extensive studies on a novel agent for preventing and/or treating a lower urinary tract symptoms, particularly dysuria with high urination efficiency. As a result, they have found that a compound having a chemical structure represented by the formula: 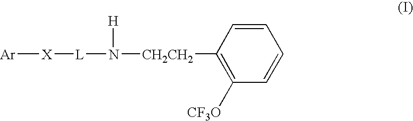
wherein Ar represents a group represented by the formula: 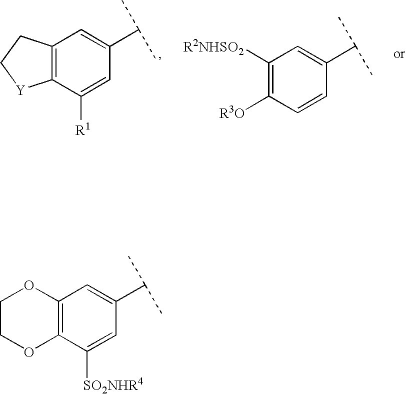
(wherein Y represents methylene or an oxygen atom, R¹ represents aminosulfonyl, C₁₋₆ alkyl-aminosulfonyl, C₁₋₆ alkyl-carbonylamino or C₁₋₆alkyl-sulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group; and L represents an optionally substituted C₄₋₅ alkylene group, or a salt thereof (hereinafter, sometimes, abbreviated as compound (I)) has, based on its structure, an unexpectedly high effect of improving excretion of the urinary bladder (effect of improving the flow rate of urine and the urination efficiency) as well as a combined effect of an acetylcholinesterase inhibitory action and an α₁ receptor antagonistic action, without having an influence on urination pressure and blood pressure. The present invention has been completed based on these findings.

That is, the present invention relates to:

[1] A compound represented by the formula: 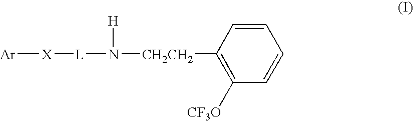
wherein Ar represents a group represented by the formula: 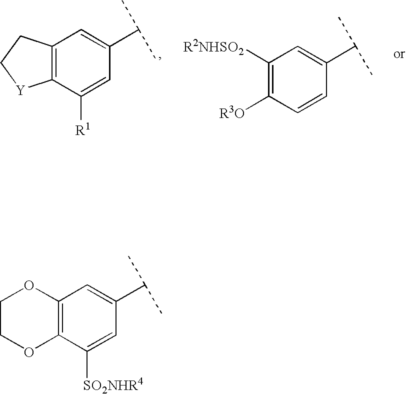
(wherein Y represents methylene or an oxygen atom, R¹ represents aminosulfonyl, C₁₋₆ alkyl-aminosulfonyl, C₁₋₆ alkyl-carbonylamino or C₁₋₆ alkyl-sulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group, and L represents an optionally substituted C₄₋₅ alkylene group, or a salt thereof;
[2] 6-[5-({2-[2-(Trifluoromethoxy)phenyl]ethyl}amino)-pentanoyl]indane-4-sulfonamide or a salt thereof;
[3] 5-[5-({2-[2-(Trifluoromethoxy)phenyl]ethyl}amino)-pentanoyl]-2,3-dihydro-1-benzofuran-7-sulfonamide or a salt thereof;
[4] N-{5-[5-({2-[2-(Trifluoromethoxy)phenyl]ethyl}amino)-pentanoyl]-2,3-dihydro-1-benzofuran-7-yl}methanesulfonamide or a salt thereof;
[5] 5-[1-hydroxy-5-({2-[2-(Trifluoromethoxy)phenyl]ethyl}-amino)pentyl]-2,3-dihydro-1-benzofuran-7-sulfonamide or a salt thereof;
[6] Crystals of a salt of 5-[5-({2-[2-(trifluoromethoxy)-phenyl]ethyl}amino)pentanoyl]-2,3-dihydro-1-benzofuran-7-sulfonamide having a melting point of 90° C. or higher;
[7] Crystals of 5-[5-({2-[2-(trifluoromethoxy)phenyl]-ethyl}amino)pentanoyl]-2,3-dihydro-1-benzofuran-7-sulfonamide p-toluene sulfonate;
[8] Crystals of 5-[5-({2-[2-(trifluoromethoxy)phenyl]-ethyl}amino)pentanoyl]-2,3-dihydro-1-benzofuran-7-sulfonamide p-toluene sulfonate having a melting point of about 153° C. to about 163° C.;
[9] A method for preparing 5-[5-({2-[2-(trifluoromethoxy)-phenyl]ethyl}amino)pentanoyl]-2,3-dihydro-1-benzofuran-7-sulfonamide or a salt thereof, comprising reacting a compound represented by the formula: 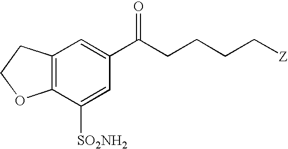
(wherein Z represents a leaving group), or a salt thereof with 2-[2-(trifluoromethoxy)phenyl]ethylamine or a salt thereof under dehydration conditions and hydrolyzing the resulting product;
[10] A pharmaceutical composition comprising a compound represented by the formula: 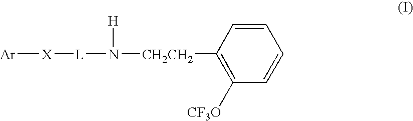
wherein Ar represents a group represented by the formula: 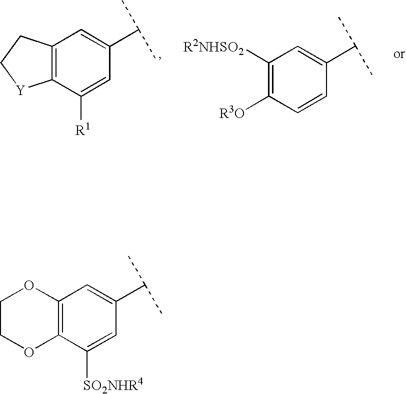
(wherein Y represents methylene or an oxygen atom, R¹ represents aminosulfonyl, C₁₋₆ alkyl-aminosulfonyl, C₁₋₆ alkyl-carbonylamino or C₁₋₆ alkyl-sulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group, and L represents an optionally substituted C₄₋₅ alkylene group, or a salt thereof, or a prodrug thereof;
[11] The pharmaceutical composition as described in [10], having a combined effect of an acetylcholinesterase inhibitory action and an α₁ receptor antagonistic action;
[12] The pharmaceutical composition as described in [10], which is an agent for preventing and/or treating lower urinary tract symptoms;
[13] The pharmaceutical composition as described in [10], which is an agent for preventing and/or treating lower urinary tract symptoms accompanied by prostatic hyperplasia;
[14] The pharmaceutical composition as described in [10], which is an agent for preventing and/or treating lower urinary tract symptoms by hypotonic bladder;
[15] A method for preventing and/or treating lower urinary tract symptoms, comprising administering an effective amount of a compound represented by the formula: 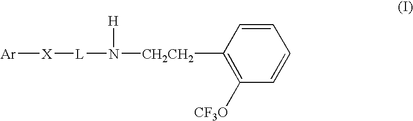
wherein Ar represents a group represented by the formula: 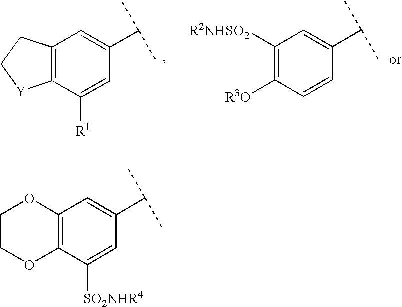
(wherein Y represents methylene or an oxygen atom, R¹ represents aminosulfonyl, C₁₋₆ alkyl-aminosulfonyl, C₁₋₆ alkyl-carbonylamino or C₁₋₆ alkyl-sulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group, and L represents an optionally substituted C₄₋₅ alkylene group, or a salt thereof, or a prodrug thereof to a mammal;
[16] Use of a compound represented by the formula: 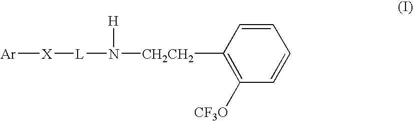
wherein Ar represents a group represented by the formula: 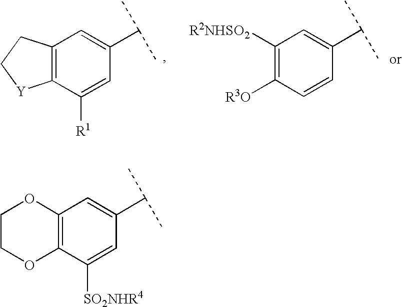
(wherein Y represents methylene or an oxygen atom, R¹ represents aminosulfonyl, C₁₋₆ alkyl-aminosulfonyl, C₁₋₆ alkyl-carbonylamino or C₁₋₆ alkyl-sulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group, and L represents an optionally substituted C₄₋₅ alkylene group, or a salt thereof, or a prodrug thereof in the preparation of an agent for preventing and/or treating lower urinary tract symptoms; and the like.

The compound of the present invention has a combined effect of an acetylcholinesterase inhibitory action and an α₁ receptor antagonistic action, thus having a high effect of improving the excretion function of the urinary bladder (effect of improving the flow rate of urine and the urination efficiency), while not affecting the urination pressure and the blood pressure, thus it being useful as an agent for preventing and/or treating lower urinary tract symptoms.

### BEST MODE FOR CARRYING OUT THE INVENTION

Each of the symbols in the formula of compound (I) is described as follows:

Ar represents a group represented by the formula: 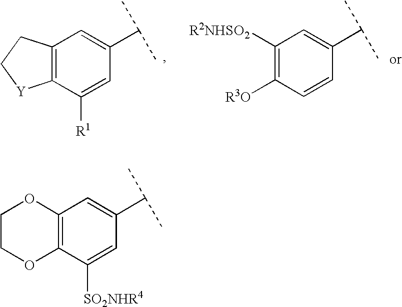
wherein Y represents methylene or an oxygen atom, R¹ represents aminosulfonyl, C₁₋₆ alkyl-aminosulfonyl, C₁₋₆ alkyl-carbonylamino or C₁₋₆ alkyl-sulfonylamino, R² represents a hydrogen atom or C₁₋₆ alkyl, R³ represents C₁₋₆ alkyl, and R⁴ represents a hydrogen atom or C₁₋₆ alkyl.

The “C₁₋₆ alkyl-aminosulfonyl” represented by R¹ includes, for example, methylaminosulfonyl, ethylaminosulfonyl, propylaminosulfonyl, isopropylaminosulfonyl, butylaminosulfonyl, isobutylaminosulfonyl, sec-butylaminosulfonyl, tert-butylaminosulfonyl, pentylaminosulfonyl, hexylaminosulfonyl, and the like.

The “C₁₋₆ alkyl-carbonylamino” represented by R¹ includes, for example, methylcarbonylamino, ethylcarbonylamino, propylcarbonylamino, isopropylcarbonylamino, butylcarbonylamino, isobutylcarbonylamino, sec-butylcarbonylamino, tert-butylcarbonylamino, pentylcarbonylamino, hexylcarbonylamino, and the like.

The “C₁₋₆ alkyl-sulfonylamino” represented by R includes, for example, methylsulfonylamino, ethylsulfonylamino, propylsulfonylamino, isopropylsulfonylamino, butylsulfonylamino, isobutylsulfonylamino, sec-butylsulfonylamino, tert-butylsulfonylamino, pentylsulfonylamino, hexylsulfonylamino, and the like.

The “C₁₋₆ alkyl” represented by R², R³ and R⁴ includes, for example, methyl, ethyl, propyl, isopropyl, butyl, isobutyl, sec-butyl, tert-butyl, pentyl, hexyl, and the like.

The “optionally substituted C₄₋₅ alkylene group” represented by L includes, for example, C₄₋₅ alkylene group (—CH₂CH₂CH₂CH₂—, or —CH₂CH₂CH₂CH₂CH₂—), which may be substituted with 1 to 5, preferably 1 to 3 substituents selected from a halogen atom (e.g., fluorine, chlorine, bromine, iodine, etc.), oxo, C₁₋₃ alkylenedioxy (e.g., methylenedioxy, ethylenedioxy, etc.), nitro, cyano, optionally halogenated C₁₋₆ alkoxy (e.g., methoxy, ethoxy, propoxy, isopropoxy, butoxy, tert-butoxy, etc.), optionally halogenated C₁₋₆ alkylthio (e.g., methylthio, ethylthio, propylthio, isopropylthio, butylthio, tert-butylthio, etc.), hydroxy, amino, mono- or di- C₁₋₆ alkylamino (e.g., methylamino, ethyl amino, dimethylamino, diethyl amino, etc.), formyl, carboxy, carbamoyl, thiocarbamoyl, optionally halogenated C₁₋₆ alkyl-carbonyl (e.g., methylcarbonyl, ethylcarbonyl, propylcarbonyl, isopropylcarbonyl, butylcarbonyl, isobutylcarbonyl, tert-butylcarbonyl, etc.), C₁₋₆ alkoxycarbonyl (e.g., methoxycarbonyl, ethoxycarbonyl, propoxycarbonyl, isopropoxycarbonyl, butoxycarbonyl, tert-butoxycarbonyl, etc.), mono- or di-C₁₋₆ alkyl-carbamoyl (e.g., methylcarbamoyl, ethylcarbamoyl, dimethylcarbamoyl, diethylcarbamoyl, etc.), optionally halogenated C₁₋₆ alkylsulfonyl (e.g., methylsulfonyl, ethylsulfonyl, propylsulfonyl, isopropylsulfonyl, butylsulfonyl, isobutylsulfonyl, tert-butylsulfonyl, etc.), formylamino, C₁₋₆ alkylsulfonylamino (e.g., methylsulfonylamino, ethylsulfonylamino, propylsulfonylamino, isopropylsulfonylamino, butylsulfonylamino, isobutylsulfonylamino, tert-butylsulfonylamino, etc.), C₁₋₆ alkylcarbonyloxy (e.g., methylcarbonyloxy, ethylcarbonyloxy, propylcarbonyloxy, isopropylcarbonyloxy, butylcarbonyloxy, isobutylcarbonyloxy, tert-butylcarbonyloxy, etc.), C₁₋₆ alkoxycarbonyloxy (e.g., methoxycarbonyloxy, ethoxycarbonyloxy, propoxycarbonyloxy, isopropoxycarbonyloxy, butoxycarbonyloxy, tert-butoxycarbonyloxy, etc.), mono- or di- C₁₋₆ alkylcarbamoyloxy (e.g., methylcarbamoyloxy, ethylcarbamoyloxy, dimethylcarbamoyloxy, diethylcarbamoyloxy, etc.), phenyl, and the like.

For Ar, the group represented by the formula: 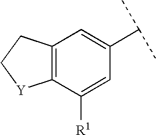
wherein Y represents methylene or an oxygen atom, and R¹ represents aminosulfonyl, C₁₋₆ alkyl-aminosulfonyl, C₁₋₆ alkyl-carbonylamino or C₁₋₆ alkyl-sulfonylamino, is preferable, and the group represented by the formula: 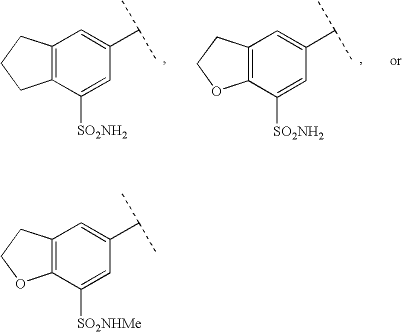
is particularly preferable.

For X, either of a carbonyl group and a methylene group which may be substituted with a hydroxy group is preferable.

For L, an unsubstituted C₄₋₅ alkylene group —CH₂CH₂CH₂CH₂—, —CH₂CH₂CH₂CH₂CH₂—, in particular, —CH₂CH₂CH₂CH₂— is preferable.

If compound (I) is in the form of a salt, examples of the salt include, for example, a salt with an inorganic acid, a salt with an organic acid, a salt with an acidic amino acid, and the like.

Preferable examples of the salt with an inorganic acid include, for example, salts with hydrochloric acid, hydrobromic acid, nitric acid, sulfuric acid, phosphoric acid, and the like.

Preferable examples of the salt with an organic acid include, for example, salt with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, and the like.

Preferable examples of the salt with an acidic amino acid include, for example, salts with aspartic acid, glutamic acid, and the like.

Among these salts, a pharmaceutically acceptable salt thereof is preferable, and an inorganic salt thereof such as hydrochloride, sulfate, phosphate, and hydrobromide; or an inorganic salt thereof such as acetate, maleate, fumarate, succinic acid salt, methanesulfonate, p-toluenesulfonate, citrate, and tartrate is preferable.

Compound (I) may be either in the anhydride form or in the hydrate form. If compound (I) is in the hydrate form, the compound may have 0.1 to 5 water molecules.

Further, compound (I) may be labeled with an isotope (e.g., ³H, ¹⁴C, ³⁵S, etc.).

If compound (I) contains an optical isomer, it can be encompassed by the compound of the present invention, and it can be individually obtained as a single product by a per se known synthesis technique or separation technique. For example, if compound (I) coexist with its optical isomers, the optical isomers isolated from the compound are also encompassed by the compound of the present invention.

The optical isomer can be prepared by a per se known method. Specifically, an optically active synthetic compound is used. Alternatively, an optical isomer is obtained by optical resolution of a final racemic mixture using an ordinary method.

Examples of the optical resolution methods include per se known methods such as a fractional recrystallization method, a chiral column method, and a diastereomer method, which are described in detail below.

1) Fractional Recrystallization Method

The method which comprises allowing a racemate to form a salt with an optically active compound (e.g., (+)-mandelic acid, (−)-mandelic acid, (+)-tartaric acid, (−)-tartaric acid, (+)-1-phenethylamine, (−)-1-phenethylamine, cinchonine, (−)-cinchonidine, brucine, etc.), separating the salt using a fractional recrystallization method, followed by, if desired, neutralizing process to obtain a free optical isomer.

2) Chiral Column Method

This method comprises subjecting a racemate or its salt to a column for separating an optical isomer (chiral column) for separation. For example, in the case of liquid chromatography, an optical isomer mixture is added to the chiral column such as ENANTIO-OVM [produced by Toso] or CHIRAL series [produced by Daicel], which is developed using water, various buffer solutions (e.g., phosphate buffer), organic solvents (e.g., ethanol, methanol, isopropanol, acetonitrile, trifluoroacetic acid, diethylamine, etc.) as single or mixed solutions, and the optical isomers are separated. Also, in the case of gas chromatography, for example, separation is conducted using a chiral column such as CP-Chirasil-DeXCB (produced by G.L. Science Co.).

3) Diastereomer Method

In this method, a racemic mixture is subjected to a chemical reaction with an optically active reagent to give a diastereomer mixture, which is separated into a single substance by an ordinary separation means (e.g., fractional recrystallization, chromatography method, etc.). This single substance is subjecting to removal of the optically active reagent part using chemical processing such as a hydrolysis reaction. For example, compound (I) is subjected to a condensation reaction with an optically active organic acid (e.g., MTPA [α-methoxy-α-(trifluoromethyl)phenylacetic acid], (−)-menthoxyacetic acid, etc.), to give the diastereomer in an ester form or an amide form, respectively. The separated diastereomer can be converted to an optical isomer of the original compound, by applying acidic hydrolysis or basic hydrolysis.

A prodrug of compound (I) is a compound which is converted to compound (I) by reactions involving enzymes and gastric acid, etc. under physiological conditions in the living body; in other words, a compound that is changed into compound (I) by enzymatically-caused oxidation, reduction and hydrolysis, and a compound that is changed into compound (I) by hydrolysis caused by gastric acid. Examples of the prodrugs of compound (I) include compounds in which amino groups of compound (I) have been acylated, alkylated, or phosphorylated [e.g. compounds in which amino groups of compound (I) have been eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated, tert-butylated, etc.]; compounds in which hydroxy groups of compound (I) have been acylated, alkylated, phosphorylated, borated (e.g., compounds in which hydroxy groups of compound (I) have been acetylated, palmitoylated, propanoylated, pivaloylated, succinylated, fumarilated, alanylated, dimethylaminomethylcarbonylated, etc.); and the like. These compounds can be produced from compound (I) using per se known methods.

Also, a prodrug of compound (I) can be a compound which is changed to compound (I) by physiological conditions, as described in pages 163 to 198 of Molecular Design, Volume 7, “Development of Drugs”, published in 1990 by Hirokawa Shoten.

Examples of compound (I) include:
6-[5-({2-[2-(trifluoromethoxy)phenyl]ethyl}amino)-pentanoyl]indane-4-sulfonamide or a salt thereof,
N-methyl-6-[5-({2-[2-(trifluoromethoxy)phenyl]ethyl}-amino)pentanoyl]indane-4-sulfonamide or a salt thereof,
5-[5-({2-[2-(trifluoromethoxy)phenyl]ethyl}amino)-pentanoyl]-2,3-dihydro-1-benzofuran-7-sulfonamide or a salt thereof,
5-[6-({2-[2-(trifluoromethoxy)phenyl]ethyl}amino)-hexanoyl]-2,3-dihydro-1-benzofuran-7-sulfonamide or a salt thereof,
N-methyl-5-[5-({2-[2-(trifluoromethoxy)phenyl]ethyl}-amino)pentanoyl]-2,3-dihydro-1-benzofuran-7-sulfonamide or a salt thereof,
N-isopropyl-5-[5-({2-[2-(trifluoromethoxy)phenyl]-ethyl}amino)pentanoyl]-2,3-dihydro-1-benzofuran-7-sulfonamide or a salt thereof,
2-methoxy-5-[5-({2-[2-(trifluoromethoxy)phenyl]-ethyl}amino)pentanoyl]benzene sulfonamide or a salt thereof,
N-isopropyl-2-methoxy-5-[5-({2-[2-(trifluoromethoxy)-phenyl]ethyl}amino)pentanoyl]benzene sulfonamide or a salt thereof,
7-[5-({2-[2-(trifluoromethoxy)phenyl]ethyl}amino)-pentanoyl]-2,3-dihydro-1,4-benzodioxine-5-sulfonamide or a salt thereof,
N-{5-[5-({2-[2-(trifluoromethoxy)phenyl]ethyl}amino)-pentanoyl]-2,3-dihydro-1-benzofuran-7-yl}acetamide or a salt thereof,
N-{5-[5-({2-[2-(trifluoromethoxy)phenyl]ethyl}-amino)pentanoyl]-2,3-dihydro-1-benzofuran-7-yl}methanesulfonamide or a salt thereof,
5-[1-hydroxy-5-({2-[2-(trifluoromethoxy)phenyl]-ethyl}amino)pentyl]-2,3-dihydro-1-benzofuran-7-sulfonamide or a salt thereof, and the like, and preferably
6-[5-({2-[2-(trifluoromethoxy)phenyl]ethyl}amino)-pentanoyl]indane-4-sulfonamide or a salt thereof,
5-[5-({2-[2-(trifluoromethoxy)phenyl]ethyl}-amino)pentanoyl]-2,3-dihydro-1-benzofuran-7-sulfonamide or a salt thereof,
N-{5-[5-({2-[2-(trifluoromethoxy)phenyl]ethyl}amino)-pentanoyl]-2,3-dihydro-1-benzofuran-7-yl}methanesulfonamide or a salt thereof,
5-[1-hydroxy-5-({2-[2-(trifluoromethoxy)phenyl]ethyl}-amino)pentyl]-2,3-dihydro-1-benzofuran-7-sulfonamide or a salt thereof, and the like. Among them,
5-[5-({2-[2-(trifluoromethoxy)phenyl]ethyl}amino)-pentanoyl]-2,3-dihydro-1-benzofuran-7-sulfonamide or a salt thereof, and particularly,
5-[5-({2-[2-(trifluoromethoxy)phenyl]ethyl}amino)-pentanoyl]-2,3-dihydro-1-benzofuran-7-sulfonamide p-toluene sulfonate is preferable.

Compound (I) may be in the crystal form.

The crystals of compound (I) can be prepared by crystallization using a per se known method for crystallization.

Examples of the method for crystallization include, for example, crystallization from a solution, crystallization from a vapor, crystallization from a melt, and the like.

The method for said “crystallization from a solution” is typically a method of shifting a non-saturated state to supersaturated state by varying factors involved in solubility of compounds (solvent composition, pH, temperature, ionic strength, redox state etc.) or the amount of solvent. The concrete examples include a concentration method, a slow cooling method, a reaction method (diffusion method or electrolysis method), a hydrothermal formation method, a fluxing agent method and the like. Examples of the solvent to be used include aromatic hydrocarbons (e.g., benzene, toluene, xylene, etc.), halogenated hydrocarbons (e.g., dichloromethane, chloroform, etc.), saturated hydrocarbons (e.g., hexane, heptane, cyclohexane, etc.), ethers (e.g., diethyl ether, diisopropyl ether, tetrahydrofuran, dioxane, etc.), nitrites (e.g., acetonitrile, etc.), ketones (e.g., acetone, etc.), sulfoxides (e.g., dimethyl sulfoxide, etc.), acid amides (e.g., N,N-dimethylformamide, etc.), esters (e.g., ethyl acetate, etc.), alcohols (e.g., methanol, ethanol, isopropyl alcohol, etc.), water and the like. These solvents are used alone or in combination of two or more thereof in an adequate ratio (for example, 1:1 to 1:100). Seed crystals can be used, if necessary.

Examples of the method for said “crystallization from a vapor” include an evaporation method (a sealed tube method or an air stream method), a vapor phase reaction method, a chemical transportation method, and the like.

Examples of the method for said “crystallization from a melt” include a normal freezing method (pulling-up method, temperature gradient method or Bridgman method), a zone melting method (zone leveling method or float zone method), a special growth method (VLS method or liquid-phase epitaxy method), and the like.

Preferable examples of the method for crystallization include a method comprising dissolving compound (I) in a suitable solvent (e.g., alcohols such as methanol, ethanol, etc.) at 20 to 120° C., and then cooling the obtained solution to no higher than the temperature for dissolution (for example, 0 to 50° C., preferably 0 to 20° C.), and other methods.

Thus obtained crystals of the present invention can be isolated, for example, by filtration.

As for the method for analyzing the thus-obtained crystals, generally, crystal analysis by a X-ray diffraction method is employed. Furthermore, a method for determining the orientation of crystals is exemplified by a mechanical method, an optical method or the like.

The crystals of compound (I) obtained by the above-described preparation method (hereinafter, abbreviated as “the crystals of the present invention) has high purity, high quality, low moisture absorbency, and reduced denaturation during long-term storage under a normal condition, and extremely excellent stability. Further, it is excellent in biological properties (e.g., pharmacokinetics (absorbency, distribution, metabolism, or excretion), exhibition of pharmaceutical efficacy, etc.), and is thus very useful for a pharmaceutical composition.

For the crystals of the present invention, the crystals of a salt of 5-[5-({2-[2-(trifluoromethoxy)phenyl]ethyl}amino)pentanoyl]-2,3-dihydro-1-benzofuran-7-sulfonamide are preferably used.

The crystals of a salt of 5-[5-({2-[2-(trifluoromethoxy)phenyl]ethyl}amino)pentanoyl]-2,3-dihydro-1-benzofuran-7-sulfonamide preferably have a melting point of 90° C. or higher, and examples thereof include crystals of a 5-[5-({2-[2-(trifluoromethoxy)phenyl]ethyl}amino)pentanoyl]-2,3-dihydro-1-benzofuran-7-sulfonamide p-toluene sulfonate, the crystal of 5-[5-({2-[2-(trifluoromethoxy)phenyl]ethyl}amino)pentanoyl]-2,3-dihydro-1-benzofuran-7-sulfonamide hydrochloride, the crystals of 5-[5-({2-[2-(trifluoromethoxy)phenyl]ethyl}amino)pentanoyl]-2,3-dihydro-1-benzofuran-7-sulfonamide methane sulfonate, crystals of 5-[5-({2-[2-(trifluoromethoxy)phenyl]ethyl}amino)pentanoyl]-2,3-dihydro-1-benzofuran-7-sulfonamide fumarate, and the like.

In particular, crystals of salt of 5-[5-({2-[2-(trifluoromethoxy)phenyl]ethyl}amino)pentanoyl]-2,3-dihydro-1-benzofuran-7-sulfonamide has preferably a melting point of about 153 to about 163° C., and one preferable example thereof is the crystal of 5-[5-({2-[2-(trifluoromethoxy)phenyl]ethyl}amino)pentanoyl]-2,3-dihydro-1-benzofuran-7-sulfonamide p-toluene sulfonate.

Further, 5-[5-({2-[2-(trifluoromethoxy)phenyl]ethyl}amino)pentanoyl]-2,3-dihydro-1-benzofuran-7-sulfonamide p-toluene sulfonate indicates a diffraction pattern having characteristic peaks at surface spacings (d values) of about 25.4, about 12.8, about 11.2, about 8.56, about 6.42, about 5.32, about 5.13, about 4.44, and about 4.28 Angstroms.

Crystals of 5-[5-({2-[2-(trifluoromethoxy)phenyl]ethyl}amino)pentanoyl]-2,3-dihydro-1-benzofuran-7-sulfonamide p-toluene sulfonate can be obtained by the “method for crystallization” as exemplified regarding compound (I), but a concentration method using alcohols, ketones, esters, or water, and a slow cooling method is preferably applied.

The crystals of 5-[5-({2-[2-(trifluoromethoxy)phenyl]ethyl}amino)pentanoyl]-2,3-dihydro-1-benzofuran-7-sulfonamide p-toluene sulfonate have high purity (purity: 99% or higher), high quality, low moisture absorbency, and reduced denaturation during long-term storage under a normal condition, and extremely excellent stability. Further, it is excellent in biological properties (e.g., pharmacokinetics (absorbency, distribution, metabolism, or excretion), exhibition of pharmaceutical efficacy, etc.), and has a low risk of toxicity (HERG inhibition, phototoxicity, etc.), thus it being very useful for a pharmaceutical composition.

The melting point as used herein means, for example, a melting point as measured using a melting point apparatus (Yanaco, MP-500 D type) or a DSC (differential scanning calorimetry) apparatus (SEIKO, EXSTAR6000), or the like.

The peak by the powder X-ray diffraction as used herein means, for example, a peak as measured by RINT Ultima⁺ 2100 type (Rigakudenki Corp.) using Cu—Kα radiation, or the like, for example, as a light source.

Generally, the melting point and the peak by the powder X-ray diffraction may vary depending on the measuring apparatus, the measuring conditions, or the like. The crystals as used herein may be crystals exhibiting different values from the melting points and the peaks by the powder X-ray diffraction as described in the present specification, within an ordinary error range.

Compound (I) can be prepared by the method as described in EP Patent Publication No. 1466625, and for example, can be prepared by the process of [Preparation process A], [Preparation Process B], or [Preparation process C], which is described below. In [Preparation process A], [Preparation Process B], or [Preparation process C], when alkylation, hydrolysis, amination, esterification, amidation, etherification, oxidation, reduction, reductive amination, or the like is carried out, these reactions are carried out in accordance with per se known methods. Examples of these methods include a method described in ORGANIC FUNCTIONAL GROUP PREPARATIONS, 2nd Ed., ACADEMIC PRESS, INC., 1989; a method described in Comprehensive Organic Transformations, VCH Publishers Inc., 1989, and the like.

Further, for the following preparation processes, each of the compounds (II), (III), (IV), (V), (VI), (VII), (VIII), (IX), (Ia), and (Ib) may form a salt. The “salt” may be the same as the “salt” in the case where “compound (I) is a salt”.

[Preparation Process A]

Process for preparing compound (I) by coupling reaction of compound (II) with compound (III). 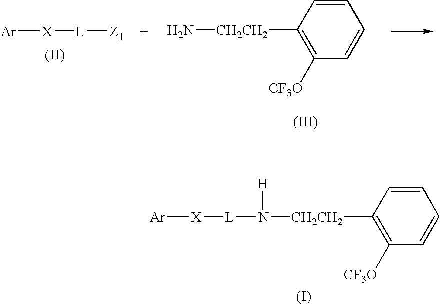
wherein Z₁ represents a leaving group, and other symbols are each as defined above.

The “leaving group” represented by Z₁ includes, for example, a halogen atom (e.g., a chlorine atom, a bromine atom, iodine atom, etc.), C₁₋₆ alkylsulfonyloxy (e.g., methanesulfonyloxy, ethanesulfonyloxy, trifluoromethanesulfonyloxy, etc.), C₆₋₁₀ arylsulfonyloxy (e.g., benzenesulfonyloxy, p-toluenesulfonyloxy, etc.), and the like. Particularly, a halogen atom (e.g., a chlorine atom, a bromine atom, etc.), methanesulfonyloxy, and the like are preferable.

The coupling reaction can be carried out without a solvent or with a suitable solvent in which the compounds are dissolved or suspended, such as a hydrocarbon solvent, an alcohol solvent, an ether solvent, a halogenated hydrocarbon solvent, an aromatic solvent, a nitrile solvent, an amide solvent, a ketone solvent, a sulfoxide solvent, a carboxylic acid solvent, water, and the like. Two or more of these solvents can be mixed at an appropriate ratio for use. Preferably, for example, no solvent is used, or an alcohol solvent such as ethanol, an aromatic solvent such as toluene, or an amide solvent such as dimethyl formamide is used.

Further, for the coupling reaction, a suitable base may be added. The base may be used as a solvent.

Examples of the “base” include:

1) strong bases exemplified by hydrides of alkali metals or alkaline earth metals (e.g., lithium hydride, sodium hydride, potassium hydride, calcium hydride, etc.), amides of alkali metals or alkaline earth metals (e.g., lithium amide, sodium amide, lithium diisopropylamide, lithium dicyclohexylamide, lithium hexamethyldisilazide, sodium hexamethyldisilazide, potassium hexamethyldisilazide, etc.), lower alkoxides of alkali metals or alkaline earth metals (e.g., sodium methoxide, sodium ethoxide, potassium tert-butoxide, etc.), etc.;

2) inorganic bases exemplified by hydroxides of alkali metals or alkaline earth metals (e.g., sodium hydroxide, potassium hydroxide, lithium hydroxide, barium hydroxide, etc.), carbonates of alkali metals or alkaline earth metals (e.g., sodium carbonate, potassium carbonate, cesium carbonate, etc.) and hydrogencarbonates of alkali metals or alkaline earth metals (e.g., sodium hydrogencarbonate, potassium hydrogencarbonate, etc.), etc.; and

3) organic bases exemplified by amines such as triethylamine, diisopropylethylamine, N-methylmorpholine, etc.; amidines such as DBU (1,8-diazabicyclo[5.4.0]undec-7-en), DBN (1,5-diazabicyclo[4.3.0]non-5-en), etc.; basic heterocyclic compounds such as pyridine, dimethylaminopyridine, imidazole, 2,6-lutidine, etc.; and the like.

Among these “bases”, for example, salts of alkali metals such as potassium carbonate, and amines such as tiethylamine and diisopropylethylamine are preferable.

In the coupling reaction, a hydrogen atom of compound (III) may be preliminarily substituted with a metal atom, for example, an alkali metal such as lithium and sodium.

The coupling reaction can be carried out at −100° C. to 300° C., preferably 0° C. to 150° C. The reaction time is, for example, 1 minute to 1 day.

The coupling reaction can be in any ratio of compound (II) to compound (III), and either one may be used as solvent.

Compound (III) is known from the documents, and a commercially available one can be used, for example, one obtained from FluoroChem (the United Kingdom).

Compound (II) can be prepared, for example, by the processes such as Friedel-Crafts reaction. 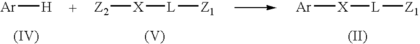
wherein Z₂ represents a leaving group, and other symbols are each as defined above.

The “leaving group” represented by Z₂ includes the same groups as the above-described Z₁, preferably, a halogen atom (e.g., a chlorine atom, a bromine atom, etc.) or a hydroxy group.

The present reaction can be preferably carried out with addition of an acid catalyst, but can be also carried out without addition of an acid catalyst. The acid catalyst used for reaction includes, for example, mineral acids such as sulfuric acid, anhydrous phosphoric acid, and polyphosphoric acid; Lewis acid such as aluminum chloride, tin tetrachloride, titanium tetrachloride, boron trifluoride, triethylaluminium, diethylaluminium chloride and zinc chloride; and the like, preferably polyphosphoric acid, aluminum chloride, diethylaluminium chloride, zinc chloride, and the like. The acid catalyst can be used in any equivalent, but usually from 0.1 equivalent to 10 equivalents based on compound (IV) or compound (V). Further, if desired, the acid catalyst can be used as a solvent.

The present reaction can be carried out without a solvent or with a suitable solvent in which the compounds are dissolved or suspended, such as a hydrocarbon solvent, an ether solvent, a halogenated hydrocarbon solvent, a nitrated solvent, an aromatic solvent, a nitrile solvent, an amide solvent, a ketone solvent, a sulfoxide solvent, a carboxylic acid solvent, and the like. Two or more of these solvents can be mixed at an appropriate ratio for use. Preferably, for example, no solvent is used, or a halogenated hydrocarbon solvent such as dichloromethane, and 1,2-dichloroethaneethan, etc.; a nitrated hydrocarbon solvent such as nitro methane, etc.; an aromatic solvent such as nitro benzene, etc.; carbon disulfide, or the like is used.

The reaction can be carried out at −100° C. to 300° C., but usually preferably 0° C. to 150° C. The reaction time is, for example, 1 minute to 3 days.

The reaction can be in any ratio of compound (IV) to compound (V), and either one may be used as solvent.

Compound (IV) can be prepared by a per se known method, or analogous methods thereto. For example, compound (IV) can be prepared by the method as described in Synthesis 10, 862 (1984), J. Chem. Soc. 1518 (1964), Synthesis 851 (1984), JP-A No. 9-124605, or the like, or analogous methods thereto.

Compound (V) can be prepared by a per se known method, or analogous methods thereto. For example, compound (IV) can be prepared by the method as described in Org. Syn. Coll. Vol. 1, 12 (1941), Helv. Chem. Acta 42, 1653 (1959), or the like, or analogous methods thereto.

[Preparation Process B]

Process for preparing compound (I) by coupling reaction of compound (VI) with compound (VII). 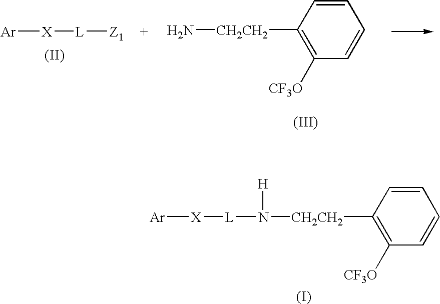
wherein each symbol is as defined above.

The coupling reaction may be carried out without a solvent or with use of a solvent. For the “solvent”, the same as for the “solvent” described in the above Preparation process A can be used, but, for example, non-solvent; or an alcohol solvent such as ethanol, etc.; an aromatic solvent such as toluene, etc., and an amidic solvent such as dimethyl formamide, etc are preferable.

Further, for the coupling reaction, a suitable base may be added. The base may be used as a solvent. For the “base”, the same base as for “base” described in the above Preparation process A can be used.

In the coupling reaction, a hydrogen atom of compound (VI) may be preliminarily substituted with a metal atom, for example, an alkali metal such as lithium and sodium, etc.

The coupling reaction can be carried out at −100° C. to 300° C., preferably 0° C. to 150° C. The reaction time is, for example, 1 minute to 1 day.

The coupling reaction can be in any ratio of compound (VI) to compound (VII), and either one may be used as solvent.

Compound (VII) can be synthesized, for example, by a process for converting the corresponding alcohol compound (VIII) as described below to a leaving group Z₁. 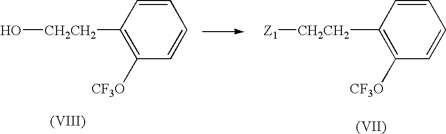
wherein each symbol is as defined above.

The leaving group Z₁ preferably includes, for example, a chlorine atom, a bromine atom, and methanesulfonyloxy. Examples of the method for converting to a chlorine atom from alcohol include, for example, the methods as described in Journal of the American Chemical Society (J. Am. Chem. Soc.) 3950 (1985), and Journal of Organic Chemistry (J. Org. Chem.) 5291 (1986). Examples of the method for converting to bromine atom from alcohol include, for example, the methods as described in Journal of the American Chemical Society (J. Am. Chem. Soc.) 1612 (1977), and Journal of the American Chemical Society (J. Am. Chem. Soc.) 8749 (1973). Examples of the method for converting to methanesulfonyloxy from alcohol include, for example, the methods as described in Journal of Medicinal Chemistry (J. Med. Chem.) 1258 (1968), and Journal of Organic Chemistry (J. Org. Chem.) 84 (1998). The alcohol compound (VIII) can be synthesized by the method as described in European Journal of Organic Chemistry (Eur. J. Org. Chem.) 691 (2001).

Compound (VI) can be prepared by subjecting compound (II) to ammonia substitution as described below. 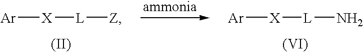
wherein each symbol is as defined above.

The present reaction can be carried out in the presence of a suitable solvent. For the “solvent”, the same as for the “solvent” described in the above Preparation process A can be used, but for example, water; an alcohol solvent such as ethanol, etc.; an aromatic solvent such as toluene, etc.; and an amidic solvent such as dimethyl formamide are preferable.

The present substitution reaction can be carried out at −100° C. to 300° C., preferably 0° C. to 200° C. If the reaction is carried out under the heating condition, a compression device such as autoclave and a sealed tube is preferably used. The reaction time is, for example, 1 minute to 1 day.

Compound (VI) can be also prepared, for example, by subjecting compound (II) to azidation, and then reduction. 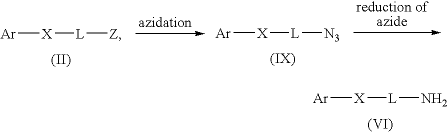
wherein each symbol is as defined above.

The azidation reaction can be carried out, for example, according to the methods as described in Journal of the American Chemical Society (J. Am. Chem. Soc.) 951 (1955), and Journal of the Chemical Society (J. Chem. Soc.) 72 (1908).

The reduction reaction of azide can be carried out, for example, according to the methods as described in Journal of Medicinal Chemistry (J. Med. Chem.) 658 (1969), and Journal of the American Chemical Society (J. Am. Chem. Soc.) 2034 (1986).

Compound (VI) can be also prepared, for example, by subjecting compound (II) to Gabriel synthesis reaction. 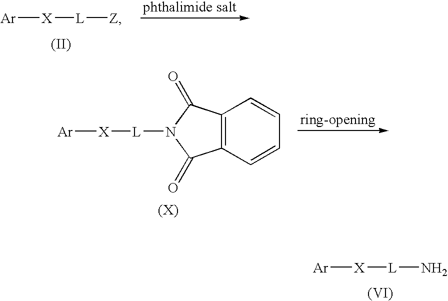
wherein each symbol is as defined above.

The Gabriel synthesis reaction can be carried out, for example, according to the methods as described in Angewandte Chemie International Edition in English (Angew. Chem. Int. Ed. Engl.) 919 (1968), and Synthesis 389 (1976).

[Preparation Process C]

If X of compound (I) is a methylene group which may be substituted with a hydroxy group, synthesis can be accomplished by subjecting the corresponding compound containing a carbonyl group to reduction reaction. That is, the reaction scheme is as follows: 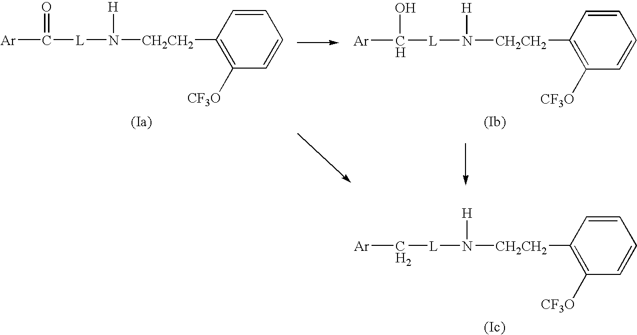
wherein each symbol is as defined above.Compound (Ib) can be prepared by reduction reaction of compound (Ia). Compound (Ic) can be further prepared by reduction reaction of compound (Ib). Alternatively, compound (Ic) can be prepared directly from compound (Ia) through reduction reaction.

The reducing agent used for the reduction reaction from compound (Ia) to compound (Ib) includes, for example, sodium borohydride, lithium borohydride, zinc borohydride, sodium cyanoborohydride, sodium triacetoxyborohydride, lithium cyanoborohydride, diisobutyl aluminum hydride, aluminum hydride, lithium aluminum hydride, borane complexes (a borane-THF complex, catecholborane, etc.), and the like, and preferably sodium borohydride, lithium aluminum hydride, and the like. The amount of the reducing agent to be used is, for example, about 0.1 to about 50 moles, preferably about 0.1 to about 10 moles based on 1 mole of compound (Ia).

The reduction reaction is carried out usually in a solvent which is inert to the reaction. The solvent includes, for example, aromatic hydrocarbons such as toluene and xylene, etc.; aliphatic hydrocarbons such as heptane and hexane, etc.; halogenated hydrocarbons such as chloroform and dichloromethane, etc.; ethers such as diethyl ether, tetrahydrofuran and dioxane, etc.; alcohols such as methanol, ethanol, 2-propanol, butanol and benzyl alcohol, etc.; nitrites such as acetonitrile, etc.; N,N-dimethyl formamide; dimethylsulfoxide; and the like. These solvents may be used in a mixture of two or more thereof in an adequate ratio.

The reaction temperature is usually about −80° C. to about 80° C., preferably about −40° C. to about 40° C., and the reaction time is usually about 5 minutes to about 48 hours, preferably about 1 hour to about 24 hours.

The reduction reaction from compound (Ia) to compound (Ib) may be carried out using the catalytic hydrogenation. The catalytic hydrogenation reaction can be carried out in the presence of a catalyst under a hydrogen atmosphere. Examples of the catalyst include palladium-based catalysts such as palladium-carbon, palladium-carbon hydroxide and palladium oxide, etc.; nickel-based catalysts such as a development nickel catalyst, etc.; platinum-based catalysts such as platinum oxide and platinum-carbon, etc.; rhodium-based catalysts such as rhodium-carbon, etc; and the like. The amount to be used is about 0.001 to about 1 mole, preferably about 0.01 to about 0.5 mole, based on 1 mole of compound (Ia).

The catalytic hydrogenation reaction is carried out usually in a solvent which is inert to the reaction. These solvents include, for example, alcohols such as methanol, ethanol, propanol and butanol, etc.; hydrocarbons such as benzene, toluene and xylene, etc.; halogenated hydrocarbons such as dichloromethane and chloroform, etc.; ethers such as diethyl ether, dioxane and tetrahydrofuran, etc.; esters such as ethyl acetate, etc.; amides such as N,N-dimethyl formamide, etc.; carboxylic acids such as acetic acid, etc.; water or a mixture thereof.

The hydrogen pressure for reaction is usually about 1 to about 50 atm, preferably about 1 to about 10 atm. The reaction temperature is usually about 0° C. to about 150° C., preferably about 20° C. to about 100° C., and the reaction time is usually about 5 minutes to about 72 hours, preferably about 0.5 hour to about 40 hours.

The reduction reaction from compound (Ib) to compound (Ic) can be carried out, for example, by the method using a reducing agent such as triethylsilane and borane, etc., or by the method using a reducing agent such as sodium borohydride and lithium aluminum hydride, in the presence of an acid (Lewis acid) such as trifluoroacetic acid, boron trifluoride, and aluminum chloride, etc. The amount of the reducing agent to be used is, for example, about 0.1 to about 50 moles, preferably about 0.1 to about 10 moles, based on 1 mole of compound (Ib).

Here, the reaction can be carried out under the conditions of the same reaction solvent, reaction temperature and reaction time as those for the above-described “reduction reaction from compound (Ia) to compound (Ib)”.

The reduction reaction from compound (Ib) to compound (Ic) may be carried out using the catalytic hydrogenation. The reaction can be carried out under the same conditions as those for the catalytic hydrogenation reaction in the above-described “reduction reaction from compound (Ia) to compound (Ib)”.

The reduction reaction from compound (Ib) to compound (Ic) preferably include, for example, a method using triethylsilane.

The reduction reaction from compound (Ia) to compound (Ic) can be carried out under the same conditions as those for the “reduction reaction from compound (Ia) to compound (Ib)” or “reduction reaction from compound (Ib) to compound (Ic)”, and can be also carried out, for example, by a method involving a Wolff-Kishner reaction as described in Organic Reactions (Org. React.) 4, 378 (1948), etc., or a method involving a Clemmensen reduction reaction as described in Organic Reactions (Org. React.) 22, 401 (1975), etc., or analogous methods thereto.

The reduction reaction from compound (Ia) to compound (Ic) preferably include, for example, a method by Wolff-Kishner reaction, or Clemmensen reduction reaction.

Among compounds (I), particularly 5-[5-({2-[2-(trifluoromethoxy)phenyl]ethyl}amino)pentanoyl]-2,3-dihydro-1-benzofuran-7-sulfonamide or a salt thereof can be synthesized according to the following preparation processes. 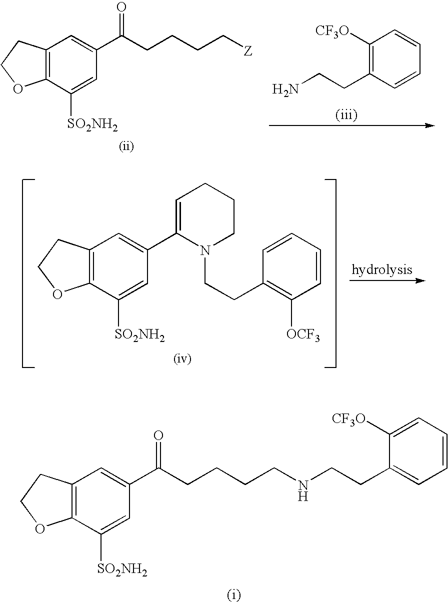
wherein Z represents a leaving group.

As the “leaving group” represented by Z, for example, a halogen atom (e.g., a chlorine atom, a bromine atom, iodine atom, etc.), C₁₋₆ alkylsulfonyloxy (e.g., methanesulfonyloxy, ethanesulfonyloxy, trifluoromethanesulfonyloxy, etc.), C₆₋₁₀ arylsulfonyloxy (e.g., benzenesulfonyloxy, p-toluenesulfonyloxy, etc.), or the like is used. Among them, a halogen atom (e.g., a chlorine atom, a bromine atom, etc.), methanesulfonyloxy, and the like are preferable, and particularly a chlorine atom is preferable.

5-[5-({2-[2-(Trifluoromethoxy)phenyl]ethyl}amino)-pentanoyl]-2,3-dihydro-1-benzofuran-7-sulfonamide (i) or a salt thereof is prepared by reacting 5-chloropentanoyl-2,3-dihydro-1-benzofuran-7-sulfonamide (ii) or a salt thereof with 2-[2-(trifluoromethoxy)phenyl]ethylamine (iii) or a salt thereof under the dehydration condition reaction, and hydrolyzing the product (iv).

The coupling reaction can be carried out without a solvent or with a suitable solvent in which the compounds are dissolved or suspended, such as a hydrocarbon solvent (e.g., benzene, toluene, hexane, heptane, etc.), an ester solvent (e.g., ethyl acetate, propyl acetate, butyl acetate, etc.), an alcohol solvent (e.g., methanol, ethanol, propanol, etc.), an ether solvent (e.g., diethyl ether, tetrahydrofuran, dioxane, etc.), a halogenated hydrocarbon solvent (e.g., dichloromethane, dichloroethane, chloroform, carbon tetrachloride, etc.), a nitrile solvent (e.g., acetonitrile, etc.), an amide solvent (e.g., dimethyl formamide, dimethylacetamide, etc.), a ketone solvent (e.g., acetone, methyl ethyl ketone, methylbutyl ketone, methyl isobutyl ketone, etc.), sulfoxide solvent (e.g., dimethylsulfoxide, etc.), a carboxylic acid solvent (e.g., acetic acid, etc.), and the like. Two or more of these solvents may be mixed at an appropriate ratio for use. Preferably, an ester solvent such as propyl acetate, a hydrocarbon solvent such as toluene, an amide solvent such as dimethyl formamide, or the like is used.

Further, for the coupling reaction, a suitable base may be added. The base may be used as a solvent.

Examples of the “base” include:

1) strong bases exemplified by hydrides of alkali metals or alkaline earth metals (e.g., lithium hydride, sodium hydride, potassium hydride, calcium hydride, etc.), amides of alkali metals or alkaline earth metals (e.g., lithium amide, sodium amide, lithium diisopropylamide, lithium dicyclohexylamide, lithium hexamethyldisilazide, sodium hexamethyldisilazide, potassium hexamethyldisilazide, etc.), lower alkoxides of alkali metals or alkaline earth metals (e.g., sodium methoxide, sodium ethoxide, potassium tert-butoxide, etc.), etc.;

2) inorganic bases exemplified by hydroxides of alkali metals or alkaline earth metals (e.g., sodium hydroxide, potassium hydroxide, lithium hydroxide, barium hydroxide, etc.), carbonates of alkali metals or alkaline earth metals (e.g., sodium carbonate, potassium carbonate, cesium carbonate, etc.) and hydrogencarbonates of alkali metals or alkaline earth metals (e.g., sodium hydrogencarbonate, potassium hydrogencarbonate, etc.), etc.; and

3) organic bases exemplified by amines such as triethylamine, diisopropylethylamine, N-methylmorpholine, etc.; amidines such as DBU (1,8-diazabicyclo[5.4.0]undec-7-en), DBN (1,5-diazabicyclo[4.3.0]non-5-en), etc.; basic heterocyclic compounds such as pyridine, dimethylaminopyridine, imidazole, 2,6-lutidine, etc.; and the like.

Among these “bases”, for example, salts of alkali metals such as potassium carbonate and sodium carbonate, and amines such as tiethylamine and diisopropylethylamine are preferable.

The coupling reaction may be performed in the presence of a salt such as sodium iodide and potassium bromide.

In the coupling reaction, compound (ii) is used in a larger amount than that of compound (iii), usually 1 to 3 moles of compound (ii) is used based on 1 mole of compound (iii). Further, a hydrogen atom on compound (iii) may be previously replaced by a metal atom, for example, an alkali metal such as lithium and sodium.

The coupling reaction can be carried out at −100° C. to 300° C., preferably 0° C. to 150° C. The reaction time is, for example, 1 minute to 1 day.

For the dehydration condition in the coupling reaction, (1) a method using a Dean-Stark device or an analogous device thereto to remove water as one of the reaction products from the reaction system, (2) a method of having a drying agent (e.g., molecular sieve) coexist in the reaction system to remove water as one of the reaction products, (3) a method of adding solvent to the reaction system intermittently to subject the reaction mixture to azeotropy to remove water as one of the reaction products, while distilling water off, or the like is used.

Compound (I) used in the present invention has a combined effect of an acetylcholinesterase inhibitory action and an α₁ antagonistic action. For the balance between the two actions, in the in vitro test, the ratio of the IC₅₀ values of the acetylcholinesterase inhibitory action and the α₁ (α_{1A}) antagonistic action is preferably, for example, about 1:1000 to about 1000:1, more preferably about 1:100 to about 100:1, even more preferably about 1:20 to about 20:1. The compound having an α₁ antagonistic action larger than the acetylcholinesterase inhibitory action, for example, the compound having the ratio of the IC₅₀ values of the acetylcholinesterase inhibitory action and the α₁ antagonistic action of about 1:1 to about 30:1, in particular about 1:1 to about 20:1 is more preferable. In addition, the balance between the two actions can be accurately estimated in the in vivo test.

Compound (I) used in the present invention has excellent activity, as well as low toxicity including HERG inhibitory action, phototoxicity; and good oral absorbency. Further, compound (I) has an effect of improving the flow rate of urine and the urination efficiency, as well as not affecting the urination pressure and the blood pressure, thus it being useful as an agent for preventing and/or treating lower urinary tract symptoms in mammals including human. For example, compound (I) can be used as an agent for preventing and/or treating lower urinary tract symptoms caused by the following 1) through 7), in particular as an agent for preventing and/or treating lower urinary tract symptoms: 1) benign prostatic hyperplasia, 2) bladder neck atresia, 3) neurogenic bladder dysfunction, 4) diabetes mellitus, 5) surgeries, 6) dertusor underactivity, and 7) Sjogren syndrome (dry eyes and mouth, cunnus drying, etc.).

More specifically, compound (I) can be used as an agent for preventing and/or treating lower urinary tract symptoms caused by a dertusor underactivity from benign prostatic hyperplasia, a dertusor underactivity from diabetes mellitus, a dertusor underactivity from diabetic neuropathy, an idiopathic dertusor underactivity (including one due to aging), a dertusor underactivity from multiple sclerosis, a dertusor underactivity from Parkinson's disease, a dertusor underactivity from spinal cord injury, a postoperative dertusor underactivity, a dertusor underactivity from cerebral infarction, a neurogenic bladder from diabetes mellitus, a neurogenic bladder from diabetic neuropathy, a neurogenic bladder from multiple sclerosis, a neurogenic bladder from Parkinson's disease, a neurogenic bladder from spinal cord injury, a neurogenic bladder from cerebral infarction, or the like.

Further, compound (I) can be used as an agent for preventing and/or treating lower urinary tract symptoms, especially urinary storage disorders such as urgency by an overactive bladder, urinary frequency, a dertusor underactivity accompanied by an overactive bladder, and incontinence.

Further, compound (I) can be used as an agent for preventing and/or treating glaucoma.

Compound (I) can be prepared, by a per se known means, into a preparation, as it is, or in a mixture with a pharmaceutically acceptable carrier at an appropriate ratio, and the preparation can be safely administered in an oral route or parenteral route (e.g., topical route, rectal route, intravenous route, and the like), such as tablets (including sugar-coated tablets and film-coated tablets), powders, granules, capsules (including soft capsules), solutions, injectable preparations, suppositories, sustained-release preparations, and the like.

Examples of the carrier used in the pharmaceutical composition, the agent for preventing and/or treating lower urinary tract symptoms of the present invention include various organic or inorganic carrier substances which are commonly used as materials for pharmaceutical preparations, such as excipients, lubricants, binders, and disintegrators in solid preparations; solvents, solubilizing agents, suspending agents, isotonizing agents, buffering agents, soothing agents, in liquid preparations; and the like. Also, in the pharmaceutical manufacturing process, additives such as antiseptics, antioxidants, coloring agents, sweeteners, absorbents, moistening agents, etc., can be used, if necessary.

Examples of the excipients include lactose, sucrose, D-mannitol, starch, cornstarch, crystalline cellulose, light anhydrous silicic acid, etc.

The lubricants include, for example, magnesium stearate, calcium stearate, talc, colloidal silica, and the like.

The binders include, for example, crystalline cellulose, refined sugar, D-mannitol, dextrin, hydroxypropyl cellulose, hydroxypropyl methyl cellulose, polyvinylpyrrolidone, starch, sucrose, gelatin, methylcellulose, sodium carboxymethylcellulose, and the like.

The disintegrators include, for example, starch, carboxymethyl cellulose, calcium carboxymethylcellulose, sodium carboxymethyl starch, L-hydroxypropyl cellulose, and the like.

The solvents include, for example, water for injections, alcohol, propylene glycol, macrogol, sesame oil, corn oil, and the like.

The solubilizing agents include, for example, polyethylene glycol, propylene glycol, D-mannitol, benzyl benzoate, ethanol, trisaminomethane, cholesterol, triethanolamine, sodium carbonate, sodium citrate, and the like.

The suspending agents include, for example, surface activators such as stearyl triethanolamine, sodium laurylsulfate, laurylaminopropionic acid, lecithin, benzalkonium chloride, benzethonium chloride, glycerin monostearate, etc.; and hydrophilic high molecular materials such as polyvinyl alcohol, polyvinylpyrrolidone, sodium carboxymethylcellulose, methylcellulose, hydroxymethyl cellulose, hydroxyethyl cellulose, hydroxypropyl cellulose, etc.

The tonicity adjusting agents include, for example, glucose, D-sorbitol, sodium chloride, glycerin, D-mannitol, and the like.

The buffering agents include, for example, buffer solutions of phosphate, acetate, carbonate, citrate, and the like.

The soothing agents include, for example, benzyl alcohol, and the like.

The preservatives include, for example, paraoxybenzoic acid esters, chlorobutanol, benzyl alcohol, phenethyl alcohol, dehydroacetic acid, sorbic acid, and the like.

The anti-oxidants include, for example, sulfites, ascorbic acid, and the like.

The content of compound (I) used in the pharmaceutical composition of the present invention, an agent for preventing and/or treating lower urinary tract symptoms is about 0.1 to about 100% by weight based on the total weight of the composition.

The dose of compound (I) used in the pharmaceutical composition of the present invention, an agent for preventing and/or treating lower urinary tract symptoms can be appropriately selected depending on the subject of administration, route of administration, disease, etc. For example, the dose per a time when the agent treating dysuria is orally administered to an adult patient (body weight: about 60 kg), is about 0.005 to 1000 mg, preferably about 0.05 to 500 mg, more preferably about 0.5 to 200 mg of an active ingredient. These amounts can be divided into one to several doses per day for administration.

Compound (I) can be used in combination with other concomitant drugs for treating lower urinary tract symptoms (e.g., voiding difficulty, etc.) or drugs which treat other diseases, but cause lower urinary tract symptoms (e.g., voiding difficulty, etc.).

Examples of the “drug for treating a disease to cause lower urinary tract symptoms” include a drug for treating prostatic hyperplasia, a drug for treating prostate cancer, a drug for treating chronic cystitis, a drug for treating constipation, a drug for treating large bowel cancer, a drug for treating uterine cancer, a drug for treating diabetes mellitus, a drug for treating cerebrovascular disorder, a drug for treating spinal cord injury, a drug for treating spinal cord neoplasm, a drug for treating multiple sclerosis, a drug for treating dementia including Alzheimer's disease, a drug for treating Parkinson's disease, a drug for treating progressive supranuclear palsy, a drug for treating Guillain-Barre Syndrome, a drug for treating acute Autonomic Nervous System disorders, a drug for treating olivopontocerebellar atrophy, a drug for treating spondylosis, and the like.

Examples of the drug for treating prostatic hyperplasia include, for example, Allylestrenol, Chlormadinone acetate, Gestonorone caproate, Nomegestrol, Mepartricin, Finasteride, PA-109, THE-320, and the like. Further, examples of the drug for treating lower urinary tract symptoms accompanied by prostatic hyperplasia include α-reductase inhibitors such as YM-31758, YM-32906, KF-20405, MK-0434, finasteride, CS-891, etc., and the like.

Examples of the drug for treating prostate cancer include, for example, Ifosfamide, Estramustine phosphate sodium, Cyproterone, Chlormadinone acetate, Flutamide, Cisplatinm, Lonidamine, Peplomycin, Leuprorelin, Finasteride, Triptorelin-DDS, Buserelin, Goserelin-DDS, Fenretinide, Bicalutamide, Vinorelbine, Nilutamide, Leuprolide-DDS, Deslorelin, Cetrorelix, Ranpirnase, Leuprorelin-DDS, Satraplatin, Prinomastat, Exisulind, Buserelin-DDS, Abarelix-DDS), and the like.

Examples of the drug for treating chronic cystitis include, for example, Flavoxate hydrochloride, and the like.

Examples of the drug for treating constipation include, for example, Sennoside A·B, Phenovalin, and the like.

Examples of the drug for treating large bowel cancer include, for example, Chromomycin A3, Fluorouracil, Tegafur, Krestin), and the like.

Examples of the drug for treating uterine cancer include, for example, Chromomycin A3, Fluorouracil, Bleomycin hydrochloride, Medroxyprogesteroneacetate, and the like.

Examples of the drug for treating diabetes include insulin sensitizers, insulin secretion enhancers, biguanides, insulins, α-glucosidase inhibitors, β3 adrenaline receptor agonists, and the like. Examples of the insulin sensitizers include pioglitazone or its salt (preferably hydrochloride), troglitazone, rosiglitazone or its salt (preferably maleate), JTT-501, GI-262570, MCC-555, YM-440, DRF-2593, BM-13-1258, KRP-297, CS-011, and the like.

Examples of the insulin secretion enhancers include sulfonylureas. Specific examples of the sulfonylureas include tolbutamide, chlorpropamide, trazamide, acetohexamide, glyclopyramide and its ammonium salt, glibenclamide, gliclazide, glimepiride, etc. Other than the above, examples of insulin secretion enhancers include repaglinide, nateglinide, KAD-1229, JTT-608, and the like.

Examples of biguamides include metformin, buformin, etc. Examples of insulins include animal insulins extracted from bovine or porcine pancreas; semi-synthetic human insulin which is enzymatically synthesized from insulin extracted from porcine pancreas; human insulin synthesized by genetic engineering, using Escherichia coli and yeast; and the like. As insulin, also employed are insulin-zinc containing 0.45 to 0.9 (w/w)% of zinc; protamine-insulin-zinc produced from zinc chloride, protamine sulfate and insulin; and the like. In addition, insulin can be an insulin fragment or derivative (e.g., INS-1, etc.).

Examples of the α-glucosidase inhibitors include acarbose, voglibose, miglitol, emiglitate, and the like.

Examples of the β3 adrenaline receptor agonists include AJ-9677, BMS-196085, SB-226552, SR-58611-A, CP-114271, L-755507, and the like.

Other than the above, examples of the “drugs for treating diabetes” include ergoset, pramlintide, leptin, BAY-27-9955, and the like.

Examples of the drug for treating cerebrovascular disorder include Nicaraven, Bencyclane fumarate, Eurnamonine, Flunarizine, Nilvadipine, Ibudilast, Argatroban, Nizofenone, Naftidrofuryl, Nicergoline, Nimodipine, Papaveroline, Alteplase, Viquidilhydrochloride, Moxisylyte, Pentoxifylline, Dihydroergotoxine mesylate, Lemildipine, Cyclandelate, Xanthinol nicotinate, Febarbamate, Cinnarizine, Memantine, Ifenprodil, Meclofenoxate hydrochloride, Ebselen, Clopidogrel, Nebracetam, Edaravone, Clinprost-DDS, Vatanidipine, Ancrod, Dipyridamole, and the like.

Examples of the drug for treating spinal cord injury include Methylprednisolone), Dural graft matrix, and the like.

Examples of the drug for treating spinal cord neoplasm include Nimustine hydrochloride, and the like.

Examples of the drug for treating multiple sclerosis include interferon-β-1b and the like.

Examples of the drug for treating dimentia including Alzheimer's disease include Aniracetam, Arginine pyroglutamate, Nefiracetam, Nimodipine, Piracetam, Propentfylline, Vinpocetine, Indeloxazine, Vitamin E, Cinepazide, Memantine, Lisuride hydrogen malate, Pramiracetam, Zuclopenthixol, Protirelin, EGB-761, Acetyl-L-carnitine, Phosphatidylserine, Nebracetam, Taltireline, Cholinealphoscerate, Ipidacrine, Talsaclidine, Cerebrolysin, Rofecoxib, ST-618, T-588, Tacrine, Physostigmine-DDS, Huperzine A, Donepezil, Rivastigmine, Metrifonate, and the like.

Examples of the drug for treating Parkinson's disease include Talipexole, Amantadine, Pergolide, Bromocriptine, Selegiline, Mazaticol hydrochloride, Memantine, Lisuride hydrogen malate, Trihexyphenidyl, Piroheptin hydrochloride, Terguride, Ropinirole, Ganglioside-GM1, Droxidopa, Riluzole, Gabergoline, Entacapone, Rasagiline, Pramipexole, L-dopa-methylester, Tolcapone, Remacemide, Dihydroergocryptine, Carbidopa, Selegiline-DDS, Apomorphine, Apomorphine-DDS, Etilevodopa, Levodopa, and the like.

Examples of the drug for treating progressive supranuclear palsy include L-dopa, carbidopa, bromocriptine, pergolide, lisuride, amitriptyline, and the like.

Examples of the drug for treating Guillain-Barre Syndrome include TRH agents such as steroids and protireline, and the like.

Examples of the drug for treating acute Autonomic Nervous System disorders include steroids, droxidopa (L-threo-DOPS), dihydroergotamine amezinium, and the like.

Examples of the drug for treating olivopontocerebellar atrophy include TRH agents, steroids or midodrine, amezinium, and the like.

Examples of the drug for treating spondylosis include anti-inflammatory sedating drug drugs, and the like.

Examples of the “drugs which treat other diseases, but cause lower urinary tract symptoms” include muscarinic antagonists such as analgesic (morphine, tramadol hydrochloride), central skeletal muscle relaxant (baclofen), butyrophenone-based neuroleptic drugs (haloperidol), increased urinary frequency/incontinence drugs (exybutynin hydrochloride, propiverine hydrochloride, tolterodine, darifenacin, YM-905/YM-537, Temiverine (NS-21), KRP-197, and trospium); smooth muscle relaxants such as flavoxate hydrochloride; muscle relaxants such as NC-1800; P2 agonists such as clenbuto; potassium channel openers such as ZD-0947, NS-8, KW-7158 and WAY-151616; PGE2 antagonists such as ONO-8711; Vanilloid receptor agonists such as resiniferatoxin and capsaicin; tachykinin antagonists such as SR-48968 (saredutant), and SB-223412 (talnerant); deltaopioid agonists; anticholinergic agents such as atropine, scopolamine, homatropine, tropicamide, cyclopentolate, scopolamine butylbromide, propantheline bromide, methylbenactyzium bromide, mepenzolate bromide, flavoxate, pyrenecevine, ipratpium bromide, trihexyphenidyl, oxybutynin, propiverine, darifenacin, tolterodine, solifenacin, temiverine, trospium chloride or a salt thereof (example, atropine sulfate, scopolamine hydrobromide, homatropine hydrobromide, cyclopentlate hydrochloride, flavoxate hydrochloride, hydrochloric acid pyrenecevine, trihexyphenidyl hydrochloride, oxybutynin chloride, tolterodine tartarate, solifenacin succinate, etc.), antispasmodic (scopolamine butylbromide, butropium bromide, tiquizium bromide, timepidium bromide, propantheline bromide), alimentary canal antiulcer drugs (Colantyl, methaphynin, cimetidine, etc.), antiparkinson agent (trihexyphenidyl hydrochloride, biperiden, mazaticol hydrochloride, levodopa, etc.), antihistamic agents (diphenhydramine, chlorpheniramine maleate, homochlorcyclizine hydrochloride, etc.), tricyclic antidepressant agents (imipramine hydrochloride, amitoriptyline hydrochloride, clomipramine hydrochloride, amoxapine, desipramine hydrochloride, etc.), phenothiazine antipsychotic drugs (chlorpromazine, propericiazine, levomepromazine, thioridazine, etc.), benzodiazepine tranquilizing agents/sleep abirritants (diazepam, chlordiazepoxide, clotiazepam, estazolam, etc.), antiarrhythmic drugs (disopyramide, etc.), vasodilator agents (hydralazine hydrochloride, etc.), brain peripheral circulation improving drugs (pentoxifylline, etc.), bronchodilator agents (theophylline, ephedrine hydrochloride, methylephedrine hydrochloride, etc.), β-adrenergic blocking drugs (propranolol hydrochloride), cold drug (Danrich), peripheral skeletal muscle relaxants (sodium dantrolene), antitubercular agents (isoniazid), and the like.

When compound (I) is used in combination with the above-described drugs, the content thereof can be appropriately selected depending on the subject of administration, the age, weight and condition of the subject of administration, administration time, administration mode, formulation, combination of the drugs, or the like. The doses for certain patients can be determined considering the age, weight, general health condition, sex, diet, administration time, uniration rate, combination of the drugs, severity of the disease to be treated of the patient, or the like.

Typically, the dose per day of a combination of compound (I) and at least one compound selected from various disease treating agents ranges from about 1/50 of a minimum recommended clinical dose up to a maximum recommended clinical dose.

The present invention will be further explained in detail below by way of Examples, Preparation Examples and Experimental Examples. However, these Examples are mere illustrative examples and do not limit the invention. Further, variations thereof are possible without departing from the scope of the present invention.

“Room temperature” in the following Reference Examples and Examples indicates normally about 0° C. to about 30° C. “%” indicates % by weight unless otherwise specifically stated.

Other symbols used herein indicate the following meanings.

s: singlet

d: doublet

t: triplet

q: quartet

dd: double-doublet

dt: double-triplet

m: multiplet

br: broad

J: coupling constant

Hz: Hertz

CDCl₃: deuterated chloroform

DMSO-d₆: deuterated dimethylsulfoxide

¹H NMR: proton nuclear magnetic resonance (generally measured as the free form of each sample in CDCl₃, and as hydrochloride in DMSO-d₆)

IR: infrared absorption spectrum

MS: mass spectrum (generally measured by electron impact ionization).

Powder X-ray diffractometry in Examples, and Reference Examples was conducted under the following conditions:

Measurement apparatus: Rigaku Corp., RINT Ultima⁺ 2100 type

Radiation source: Cu—Kα beam (λ=1.5418 Å)

Tube voltage: 40 kV

Tube current: 50 mA

Scanning speed: 6°/min

Angle of diffraction (2θ): 2 to 35°

Codes used herein for showing bases and amino acids are based on those by the IUPAC-IUB Commission on Biochemical Nomenclature or common codes in the concerned fields. Examples of these codes are shown below. Also, where some optical isomers of amino acids can exist, the L form is shown unless otherwise specified.

DNA: deoxyribonucleic acid

cDNA: complementary deoxyribonucleic acid

A: adenine

T: thymine

G: guanine

C: cytosine

ATP: adenosine triphosphate

EDTA: ethylenediamine tetraacetic acid

### REFERENCE EXAMPLE 1

### 5-Chloro-1-(2,3-dihydro-1H-inden-5-yl)pentan-1-one

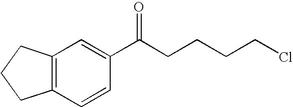

To a solution of indane (30.0 g, 84.6 mmol) and 5-chlorovalerylchloride (13.1 g, 84.6 mmol) in dichloromethane (50 mL), aluminum chloride (11.3 g, 84.7 mmol) was added by portions under water-cooling. After stirring at room temperature for 15 min, the reaction solution was poured into ice (300 g), extracted with ethyl acetate, and then washed with brine. The organic layer was dried over anhydrous magnesium sulfate, the solvent was evaporated under reduced pressure, and the residue was crystallized from hexane to obtain the titled compound as colorless crystals (15.0 g, 74%).

¹H NMR (300 MHz, CDCl₃) δ 1.80-1.95 (4H, m), 2.11 (2H, quintet, J=7.5 Hz), 2.90-3.10 (6H, m), 3.50-3.65 (2H, m), 7.28 (1H, d, J=7.8 Hz), 7.74 (1H, dd, J=7.8, 1.5 Hz), 7.80 (1H, d, J=1.5 Hz)

### REFERENCE EXAMPLE 2

### 6-(5-Chloropentanoyl)indane-4-sulfonyl chloride

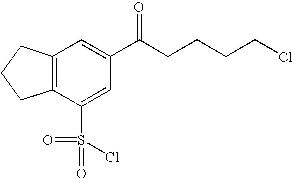

5-Chloro-1-(2,3-dihydro-1H-inden-5-yl)pentan-1-one (10.0 g, 42.2 mmol) obtained in Reference Example 1 was added to chlorosulfonic acid (50 mL) by portions under ice-cooling. The mixture was stirred at room temperature for 30 hours, and the reaction solution was added dropwise to crushed ice (500 g), extracted with ethyl acetate, and then washed with brine. The organic layer was dried over anhydrous magnesium sulfate, and then the solvent was evaporated under reduced pressure. The residue was purified by column chromatography to obtain the titled compound as a pale yellow oil (4.52 g, 32%).

¹H NMR (300 MHz, CDCl₃) δ 1.80-2.00 (4H, m), 2.27 (2H, quintet, J=7.5 Hz), 3.04 (2H, t, J=6.6 Hz), 3.09 (2H, t, J=7.5 Hz), 3.43 (2H, t, J=7.5 Hz), 3.55-3.65 (2H, m), 8.13 (1H, s), 8.34 (1H, s)

### REFERENCE EXAMPLE 3

### 6-(5-Chloropentanoyl)indane-4-sulfonamide

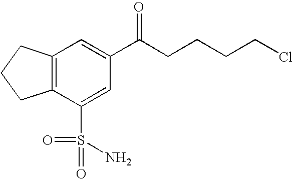

To a solution of 6-(5-chloropentanoyl)indane-4-sulfonyl chloride (3.52 g, 10.5 mmol) obtained in Reference Example 2 in tetrahydrofuran (80 mL), a 25% ammonia solution (10 mL) was added dropwise under ice-cooling. After stirring at room temperature for 15 minutes, the solvent was evaporated under reduced pressure and then the residue was extracted with ethyl acetate, and washed with brine. The organic layer was dried over anhydrous magnesium sulfate, and then the solvent was evaporated under reduced pressure to obtain the titled compound having a melting point of 117 to 119° C. as colorless crystals (2.61 g, 79%).

¹H NMR (300 MHz, CDCl₃) δ 1.80-2.00 (4H, m), 2.12 (2H, quintet, J=7.5 Hz), 2.95-3.10 (4H, m), 3.31 (2H, t, J=7.5 Hz), 3.59 (2H, t, J=6.3 Hz), 4,95 (2H, s), 8.01 (1H, s), 8.34 (1H, s)

### REFERENCE EXAMPLE 4

### 6-(5-Chloropentanoyl)-N-methylindane-4-sulfonamide

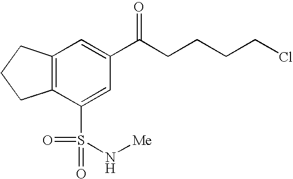

Using 6-(5-chloropentanoyl)indane-4-sulfonyl chloride (1.00 g, 2.98 mmol) obtained in Reference Example 2 and 40% methylamine in methanol (3 mL), the same procedure as in Reference Example 3 was carried out to obtain the titled compound having a melting point of 107 to 109° C. as colorless crystals (796 mg, 81%).

¹H NMR (300 MHz, CDCl₃) δ 1.80-2.00 (4H, m), 2.20 (2H, quintet, J=7.5 Hz), 2.68 (3H, d, J=5.7 Hz), 2.95-3.10 (4H, m), 3.27 (2H, t, J=7.5 Hz), 3.59 (2H, t, J=6.0 Hz), 4.55-4.65 (1H, br), 8.02 (1H, d, J=1.2 Hz), 8.25 (1H, d, J=1.2 Hz).

### REFERENCE EXAMPLE 5

### tert-Butyl 5-[7-(aminosulfonyl)-2,3-dihydro-1H-inden-5-yl]-5-oxopentyl{2-[2-(trifluoromethoxy)phenyl]ethyl}-carbamate

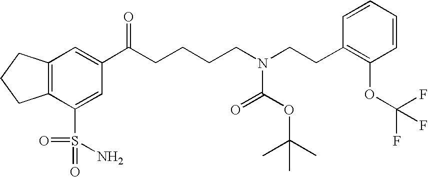

A mixture of 6-(5-chloropentanoyl)indane-4-sulfonamide (800 mg, 2.53 mmol) obtained in Reference Example 3 and {2-[2-(trifluoromethoxy)phenyl]ethyl}amine (1.04 g, 5.07 mmol) was stirred at 120° C. for 20 minutes. To the mixture, water and THF was added to give a homogeneous mixture, which was then cooled to room temperature. A solution of di-t-butyl dicarbonate (1.33 g, 6.09 mmol) in THF and then trithylamine (0.849 mL, 6.09 mmol) was added dropwise thereto, and the mixture was stirred at room temperature for 12 hours. The solvent was evaporated, and the residue was partitioned into ethyl acetate and water. The organic layer was washed with water and brine, and then dried over anhydrous magnesium sulfate. The organic layer was concentrated, and then the residue was purified by column chromatography to obtain the titled compound as a pale yellow oil (870 mg, 59%).

¹H NMR (300 MHz, CDCl₃) δ 1.37 (9H, s), 1.50-1.75 (4H, m), 2.10-2.25 (2H, m), 2.80-3.25 (8H, m), 3.29 (2H, t, J=7.5 Hz), 3.36 (2H, t, J=7.5 Hz), 5.40-5.70 (2H, br), 7.10-7.35 (4H, m), 7.96 (1H, s), 8.29 (1H, s).

### REFERENCE EXAMPLE 6

### tert-Butyl {5-[7-(aminosulfonyl)-2,3-dihydro-1H-inden-5-yl]-5-oxopentyl}[2-(2-methoxyphenyl)ethyl]carbamate

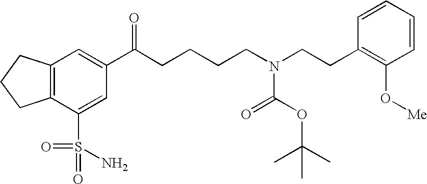

Using 6-(5-chloropentanoyl)indane-4-sulfonamide (800 mg, 2.53 mmol) obtained in Reference Example 3 and 2-(2-methoxyphenyl)ethylamine (765 mg, 5.06 mmol), the same procedure as in Reference Example 5 was carried out to obtain the titled compound as a pale yellow oil (914 mg, 68%).

¹H NMR (300 MHz, CDCl₃) δ 1.38 (9H, s), 1.40-1.95 (4H, m), 2.15 (2H, quintet, J=7.5 Hz), 2.80 (2H, t, J=7.5 Hz), 2.90-3.05 (4H, m), 3.10-3.40 (6H, m), 3.82 (3H, s), 5.20-5.60 (2H, br), 6.80-6.90 (2H, m), 7.00-7.10 (1H, m), 7.18 (1H, t, J=7.5 Hz), 7.97 (1H, s), 8.31 (1H, s).

### REFERENCE EXAMPLE 7

### tert-Butyl 5-[7-(aminosulfonyl)-2,3-dihydro-1H-inden-5-yl]-5-oxopentyl[2-(2-chlorophenyl)ethyl]carbamate

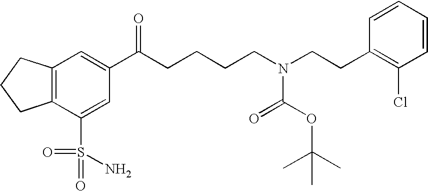

Using 6-(5-chloropentanoyl)indane-4-sulfonamide (800 mg, 2.53 mmol) obtained in Reference Example 3 and 2-(2-chlorophenyl)ethylamine (787 mg, 5.06 mmol), the same procedure as in Reference Example 5 was carried out to obtain the titled compound as a pale yellow oil (1.04 g, 77%).

¹H NMR (300 MHz, CDCl₃) δ 1.38 (9H, s), 1.40-1.85 (4H, m), 2.18 (2H, quintet, J=7.5 Hz), 2.70-3.45 (12H, m), 5.20-5.45 (2H, br), 7.10-7.40 (4H, m), 7.98 (1H, s), 8.31 (1H, s).

### REFERENCE EXAMPLE 8

### tert-Butyl 5-{7-[(methylamino)sulfonyl]-2,3-dihydro-1H-inden-5-yl}-5-oxopentyl{2-[2-(trifluoromethoxy)phenyl]ethyl}carbamate

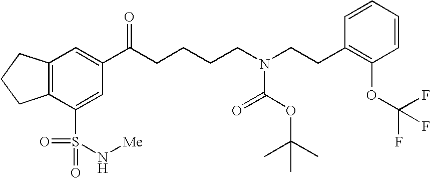

Using 6-(5-chloropentanoyl)-N-methylindane-4-sulfonamide (700 mg, 2.12 mmol) obtained in Reference Example 4 and {2-[2-(trifluoromethoxy)phenyl]ethyl}amine (870 mg, 4.24 mmol), the same procedure as in Reference Example 5 was carried out to obtain the titled compound as a pale yellow oil (570 mg, 45%).

¹H NMR (300 MHz, CDCl₃) δ 1.41 (9H, s), 1.50-1.80 (4H, m), 2.18 (2H, quintet, J=7.5 Hz), 2.66 (3H, d, J=4.8 Hz), 2.80-3.30 (8H, m), 3.26 (2H, t, J=7.5 Hz), 3.38 (2H, t, J=7.5 Hz), 4.65-4.95 (1H, br), 7.15-7.40 (4H, m), 7.98 (1H, s), 8.23 (1H, s).

### REFERENCE EXAMPLE 9

### tert-Butyl {5-[7-(aminosulfonyl)-2,3-dihydro-1-benzofuran-5-yl]-5-oxopentyl}{2-[2-(trifluoromethoxy)-phenyl]ethyl}carbamate

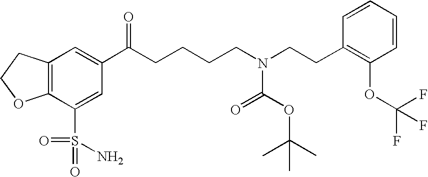

A mixture of 5-(5-chloropentanoyl)-2,3-dihydro-1-benzofuran-7-sulfonamide (20.9 g, 65.8 mmol) prepared according to the method as described in WO03-057254 and {2-[2-(trifluoromethoxy)phenyl]ethyl}amine (16.2 g, 79.0 mmol) was stirred at 130° C. for 2 hours. To the mixture, water and THF were added to obtain a homogeneous mixture, and then the solution was cooled to room temperature. A solution of di-t-butyl dicarbonate (20.7 g, 94.8 mmol) in THF was added thereto, then triethylamine (13.2 mL, 94.7 mmol) was added dropwise thereto, and the mixture was stirred at room temperature for 12 hours. The solvent was evaporated, and the residue was partitioned into ethyl acetate and water. The organic layer was washed with water and brine, and then dried over anhydrous magnesium sulfate. The organic layer was concentrated, and then the residue was purified by column chromatography, and then crystallized from ethanol-isopropyl ether to obtain the titled compound having a melting point of 123 to 124° C. as colorless crystals (19.2 g, 50%).

¹H NMR (300 MHz, CDCl₃) δ 1.40 (9H, s), 1.45-1.80 (4H, m), 2.80-3.00 (4H, m), 3.05-3.25 (2H, m), 3.25-3.40 (4H, m), 4.89 (2H, t, J=8.7 Hz), 5.22 (2H, s), 7.15-7.35 (4H, m), 8.01 (1H, s), 8.19 (1H, s).

Elemental analysis C₂₇H₃₃F₃N₂O₇S,

Calcd: C, 55.28; H, 5.67; N, 4.78.

Found: C, 55.28; H, 5.63; N, 4.67.

### REFERENCE EXAMPLE 10

### tert-Butyl {6-[7-(aminosulfonyl)-2,3-dihydro-1-benzofuran-5-yl]-6-oxohexyl}{2-[2-(trifluoromethoxy)-phenyl]ethyl}carbamate

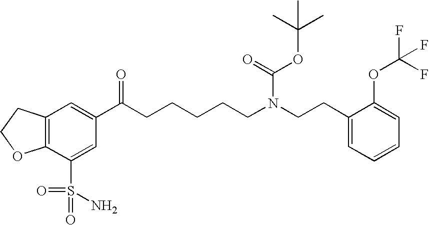

Using 5-(6-bromohexanoyl)-2,3-dihydro-1-benzofuran-7-sulfonamide (1.00 g, 3.06 mmol) prepared according to the method as described in WO 03-057254 and {2-[2-(trifluoromethoxy)phenyl]ethyl}amine (1.26 g, 6.14 mmol), the same procedure as in Reference Example 5 was carried out to obtain the titled compound as a pale yellow oil (840 mg, 46%).

¹H NMR (300 MHz, CDCl₃) δ 1.20-1.60 (4H, m), 1.39 (9H, s), 1.65-2.00 (2H, m), 2.80-2.95 (4H, m), 3.00-3.20 (2H, m), 3.25-3.40 (4H, m), 4.88 (2H, t, J=8.7 Hz), 5.25-5.45 (2H, br), 7.25-7.35 (4H, m), 7.99 (1H, s), 8.17 (1H, s).

### REFERENCE EXAMPLE 11

### tert-Butyl {6-[7-(aminosulfonyl)-2,3-dihydro-1-benzofuran-5-yl]-6-oxohexyl}[2-(2-methoxyphenyl)ethyl]-carbamate

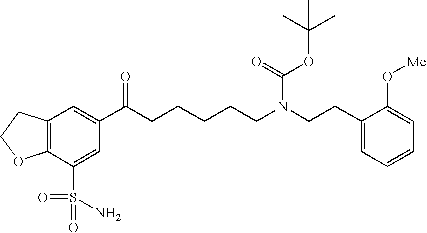

Using 5-(6-bromohexanoyl)-2,3-dihydro-1-benzofuran-7-sulfonamide (1.00 g, 3.06 mmol) prepared according to the method as described in WO03-057254 and 2-(2-methoxyphenyl)ethylamine (927 mg, 6.13 mmol), the same procedure as in Reference Example 5 was carried out to obtain the titled compound as a pale yellow oil (662 mg, 40%).

¹H NMR (300 MHz, CDCl₃) δ 1.20-1.60 (4H, m), 1.41 (9H, s), 1.65-1.80 (2H, m), 2.80 (2H, t, J=7.2 Hz), 2.89 (2H, t, J=7.2 Hz), 3.00-3.20 (2H, m), 3.25-3.40 (4H, m), 3.81 (3H, s), 4.87 (2H, t, J=8.4 Hz), 5.20-5.40 (2H, br), 6.80-6.90 (2H, m), 7.00-7.15 (1H, m), 7.18 (1H, t, J=7.8 Hz), 8.00 (1H, s), 8.18 (1H, s).

### REFERENCE EXAMPLE 12

### tert-Butyl {6-[7-(aminosulfonyl)-2,3-dihydro-1-benzofuran-5-yl]-6-oxohexyl}[2-(2-chlorophenyl)ethyl]carbamate

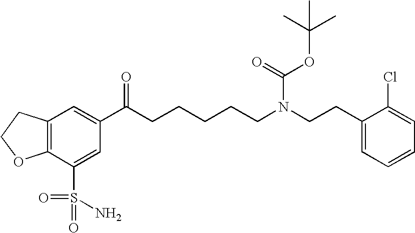

Using 5-(6-bromohexanoyl)-2,3-dihydro-1-benzofuran-7-sulfonamide (1.00 g, 3.06 mmol) prepared according to the method as described in WO03-057254 and 2-(2-chlorophenyl)ethylamine (952 mg, 6.12 mmol), the same procedure as in Reference Example 5 was carried out to obtain the titled compound as a pale yellow oil (558 mg, 33%).

¹H NMR (300 MHz, CDCl₃) δ 1.20-1.60 (4H, m), 1.39 (9H, s), 1.65-2.00 (2H, m), 2.85-3.00 (4H, m), 3.05-3, 20 (2H, m), 3.25-3.45 (4H, m), 4.88 (2H, t, J=8.7 Hz), 5.20-5.45 (2H, br), 7.05-7.40 (4H, m), 7.99 (1H, s), 8.17 (1H, s).

### REFERENCE EXAMPLE 13

### tert-Butyl (5-{7-[(methylamino)sulfonyl]-2,3-dihydro-1-benzofuran-5-yl}-5-oxopentyl){2-[2-(trifluoromethoxy)-phenyl]ethyl}carbamate

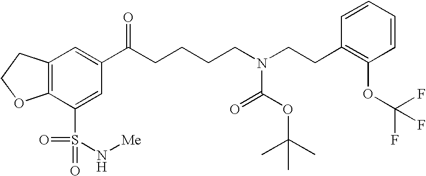

Using a mixture of 6-(5-chloropentanoyl)-N-methylindane-4-sulfonamide (1.60 g, 4.82 mmol) prepared according to the method as described in WO 03-057254 and 2-[2-(trifluoromethoxy)phenyl]ethyl}amine (1.98 g, 9.65 mmol), the same procedure as in Reference Example 5 was carried out to obtain the titled compound as a pale yellow oil (2.53 g, 87%).

¹H NMR (300 MHz, CDCl₃) δ 1.40-1.80 (4H, m), 1.42 (9H, s), 2.65 (3H, d, J=5.4 Hz), 2.80-3.00 (4H, m), 3.05-3.45 (6H, m), 4.80-5.00 (3H, m), 7.10-7.40 (4H, m), 8.04 (1H, s), 8.21 (1H, s).

### REFERENCE EXAMPLE 14

### tert-Butyl (5-{7-[(isopropylamino)sulfonyl]-2,3-dihydro-1-benzofuran-5-yl}-5-oxopentyl){2-[2-(trifluoromethoxy)phenyl]ethyl}carbamate

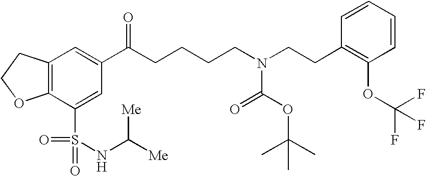

Using 5-(5-chloropentanoyl)-N-isopropyl-2,3-dihydro-1-benzofuran-7-sulfonamide (1.60 g, 4.45 mmol) prepared according to the method as described in WO 03-057254 and {2-[2-(trifluoromethoxy)phenyl]ethyl}amine (1.82 g, 8.87 mmol), the same procedure as in Reference Example 5 was carried out to obtain the titled compound as a pale yellow oil (2.28 g, 81%).

¹H NMR (300 MHz, CDCl₃) δ 1.10 (6H, d, J=6.6 Hz), 1.42 (9H, s), 1.50-1.80 (4H, m), 2.80-3.55 (11H, m), 4.72 (1H, d, J=6.6 Hz), 4.86 (2H, t, J=8.7 Hz), 7.15-7.35 (4H, m), 8.03 (1H, s), 8.23 (1H, s).

### REFERENCE EXAMPLE 15

### tert-Butyl {5-[3-(aminosulfonyl)-4-methoxyphenyl]-5-oxopentyl}{2-[2-(trifluoromethoxy)phenyl]ethyl}carbamate

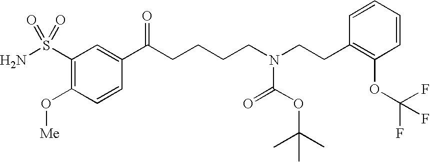

Using 5-(5-chloropentanoyl)-2-methoxybenzene sulfonamide (600 mg, 1.96 mmol) prepared according to the method as described in WO03-057254 and {2-[2-(trifluoromethoxy)phenyl]ethyl}amine (804 mg, 3.92 mmol), the same procedure as in Reference Example 5 was carried out to obtain the titled compound as a pale yellow oil (674 mg, 60%).

¹H NMR (300 MHz, CDCl₃) δ 1.30-1.75 (13H, m), 2.80-3.00 (4H, m), 3.05-3.25 (2H, m), 3.37 (2H, t, J=7.5 Hz), 4.09 (3H, s), 5.35-5.55 (2H, br), 7.11 (1H, d, J=8.7 Hz), 7.20-7.35 (4H, m), 8.15-8.20 (1H, m), 8.45 (1H, s).

### REFERENCE EXAMPLE 16

### tert-Butyl (5-{3-[(isopropylamino)sulfonyl]-4-methoxyphenyl}-5-oxopentyl){2-[2-(trifluoromethoxy)phenyl]-ethyl}carbamate

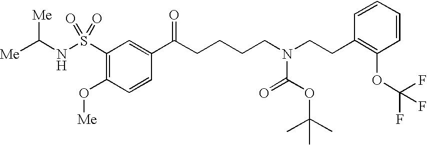

Using 5-(5-chloropentanoyl)-N-isopropyl-2-methoxybenzene sulfonamide (600 mg, 1.72 mmol) prepared according to the method as described in WO 03-057254 and {2-[2-(trifluoromethoxy)phenyl]ethyl}amine (706 mg, 3.44 mmol), the same procedure as in Reference Example 5 was carried out to obtain the titled compound as a pale yellow oil (480 mg, 45%).

¹H NMR (300 MHz, CDCl₃) δ 1.07 (6H, d, J=6.6 Hz), 1.30-1.80 (13H, m), 2.85-3.30 (6H, m), 3.35-3.50 (3H, m), 4.06 (3H, s), 4.75-4.85 (1H, br), 7.11 (1H, d, J=8.7 Hz), 7.20-7.40 (4H, m), 8.19 (1H, dd, J=8.7, 2.4 Hz), 8.50 (1H, d, J=2.7 Hz).

### REFERENCE EXAMPLE 17

### tert-Butyl {5-[8-(aminosulfonyl)-2,3-dihydro-1,4-benzodioxin-6-yl]-5-oxopentyl}{2-[2-(trifluoromethoxy)phenyl]ethyl}carbamate

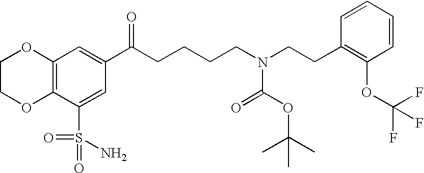

Using 7-(5-chloropentanoyl)-2,3-dihydro-1,4-benzodioxine-5-sulfonamide (500 mg, 1.50 mmol) prepared according to the method as described in WO03-057254 and {2-[2-(trifluoromethoxy)phenyl]ethyl}amine (655 mg, 3.00 mmol), the same procedure as in Reference Example 5 was carried out to obtain the titled compound as a pale yellow oil (662 mg, 73%).

¹H NMR (300 MHz, CDCl₃) δ 1.38 (9H, s), 1.40-1.75 (4H, m), 2.80-3.25 (6H, m), 3.37 (2H, t, J=7.5 Hz), 4.30-4.40 (2H, m), 4.50-4.60 (2H, m), 5.20-5.50 (2H, br), 7.10-7.35 (4H, m), 7.67 (1H, s), 8.01 (1H, s).

### REFERENCE EXAMPLE 18

### 5-Chloro-1-(7-nitro-2,3-dihydro-1-benzofuran-5-yl)pentan-1-one

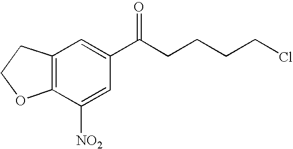

5-chloro-1-(2,3-dihydro-1-benzofuran-5-yl)-1-pentanone (10.0 g, 42.2 mmol) prepared according to the method as described in WO03-057254 was added portionwise to a mixed solution of a concentrated nitric acid solution (25 mL) and a concentrated sulfuric acid (25 mL), which were cooled with dry ice-acetone bath. The mixture was stirred in a dry ice-acetone bath for 20 minutes and quenched with water, and then the precipitated were to obtain by filtration. The obtained solid was dissolved in ethyl acetate, and washed with a potassium carbonate solution and brine. The organic layer was dried over anhydrous magnesium sulfate, and then the solvent was evaporated under reduced pressure to obtain the titled compound having a melting point of 91 to 92° C. as pale yellow crystals (7.86 g, 66%).

¹H NMR (300 MHz, CDCl₃) δ 1.80-2.00 (4H, m), 3.01 (2H, t, J=6.6 Hz), 3.38 (2H, t, J=8.7 Hz), 3.59 (2H, t, J=6.6 Hz), 4.95 (2H, t, J=8.7 Hz), 8.08 (1H, d, J=1.2 Hz), 8.53 (1H, d, J=1.2 Hz).

### REFERENCE EXAMPLE 19

### 1-(7-Amino-2,3-dihydro-1-benzofuran-5-yl)-5-chloropentan-1-one

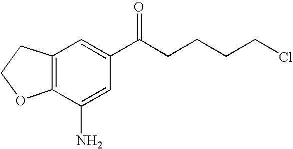

To a suspension of 5-chloro-1-(7-nitro-2,3-dihydro-1-benzofuran-5-yl)pentan-1-one (5.00 g, 17.4 mmol) obtained in Reference Example 18 in concentrated hydrochloric acid (5 mL)-acetic acid (50 mL) was added iron powder (5.00 g), and the mixture was stirred at 80° C. for 20 minutes. The solid was filtered off, the filtrate was neutralized with a potassium carbonate solution, and then the precipitant was filtered off again. The filtrate was extracted with ethyl acetate, and washed with brine. The organic layer was dried over anhydrous magnesium sulfate, and then the solvent was evaporated under reduced pressure to obtain the titled compound having a melting point of 129 to 131° C. as pale gray crystals (2.95 g, 67%).

¹H NMR (300 MHz, CDCl₃) δ 1.80-1.95 (4H, m), 2.85-2.95 (2H, m), 3.24 (2H, t, J=8.4 Hz), 3.40-3.80 (4H, m), 4.67 (2H, t, J=8.4 Hz), 7.22 (1H, d, J=1.8 Hz), 7.25-7.35 (1H, m).

### REFERENCE EXAMPLE 20

### N-[5-(5-Chloropentanoyl)-2,3-dihydro-1-benzofuran-7-yl]acetamide

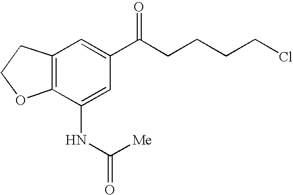

1-(7-Amino-2,3-dihydro-1-benzofuran-5-yl)-5-chloropentan-1-one (2.00 g, 7.88 mmol) obtained in Reference Example 19 was added to a mixed solution of acetic anhydride (10 mL) and pyridine (20 mL), and the mixture was stirred at room temperature for 12 hours. The solvent was evaporated, and the residue was partitioned into ethyl acetate and water. The organic layer was sequentially washed with 1 N hydrochloric acid, a potassium carbonate solution, and brine, dried over anhydrous magnesium sulfate, and then concentrated under reduced pressure to obtain the titled compound having a melting point of 150 to 152° C. as colorless crystals (1.90 g, 82%).

¹H NMR (300 MHz, CDCl₃) δ 1.80-1.95 (4H, m), 2.21 (3H, s), 2.90-3.00 (2H, m), 3.29 (2H, t, J=8.7 Hz), 3.55-3.60 (2H, m), 4.71 (2H, t, J=8.7 Hz), 7.20-7.40 (1H, br), 7.65 (1H, s), 8.73 (1H, s).

### REFERENCE EXAMPLE 21

### N-[5-(5-Chloropentanoyl)-2,3-dihydro-1-benzofuran-7-yl]methane sulfonamide

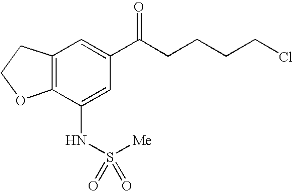

To a solution of 1-(7-amino-2,3-dihydro-1-benzofuran-5-yl)-5-chloropentan-1-one (2.00 g, 7.88 mmol) obtained in Reference Example 19 and triethylamine (1.15 mL, 8.25 mmol) in THF (50 mL), methanesulfonyl chloride (0.64 mL, 8.27 mmol) were added dropwise at room temperature, and the mixture was stirred at room temperature for 12 hours. The solvent was evaporated, and the residue was partitioned into ethyl acetate and water. The organic layer was washed with water and brine, and then dried over anhydrous magnesium sulfate. The organic layer was concentrated, and then the residue was purified by column chromatography to obtain the titled compound as colorless crystals (1.60 g, 61%).

¹H NMR (300 MHz, CDCl₃) δ 1.80-1.95 (4H, m), 2.90-3.00 (2H, m), 3.04 (3H, s), 3.34 (2H, t, J=8.7 Hz), 3.55-3.60 (2H, m), 4.75 (2H, t, J=8.7 Hz), 6.37 (1H, s), 7.73 (1H, d, J=1.2 Hz), 7.92 (1H, d, J=1.2 Hz).

### REFERENCE EXAMPLE 22

### tert-Butyl {5-[7-(acetylamino)-2,3-dihydro-1-benzofuran-5-yl]-5-oxopentyl}{2-[2-(trifluoromethoxy)-phenyl]ethyl}carbamate

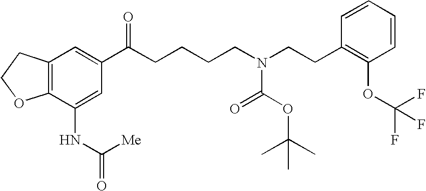

Using N-[5-(5-chloropentanoyl)-2,3-dihydro-1-benzofuran-7-yl]acetamide (800 mg, 2.70 mmol) obtained in Reference Example 20 and {2-[2-(trifluoromethoxy)phenyl]ethyl}amine (1.11 g, 5.41 mmol), the same procedure as in Reference Example 5 was carried out to obtain the titled compound as a pale yellow oil (1.18 g, 77%).

¹H NMR (300 MHz, CDCl₃) δ 1.41 (9H, s), 1.45-1.80 (4H, m), 2.20 (3H, s), 2.80-3.00 (4H, m), 3.05-3.25 (2H, m), 3.28 (2H, t, J=8.7 Hz), 3.39 (2H, t, J=7.2 Hz), 4.69 (2H, t, J=8.7 Hz), 7.20-7.40 (5H, m), 7.64 (1H, s), 8.69 (1H, s).

### REFERENCE EXAMPLE 23

### tert-Butyl (5-{7-[(methylsulfonyl)amino]-2,3-dihydro-1-benzofuran-5-yl}-5-oxopentyl){2-[2-(trifluoromethoxy)phenyl]ethyl}carbamate

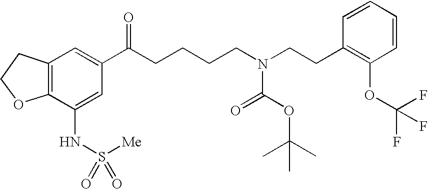

Using N-[5-(5-chloropentanoyl)-2,3-dihydro-1-benzofuran-7-yl]methanesulfonamide (800 mg, 2.41 mmol) obtained in Reference Example 21 and {2-[2-(trifluoromethoxy)phenyl]ethyl}amine (984 mg, 4.80 mmol), the same procedure as in Reference Example 5 was carried out to obtain the titled compound as a colorless oil (1.21 g, 84%).

¹H NMR (300 MHz, CDCl₃) δ 1.41 (9H, s), 1.45-1.75 (4H, m), 2.80-3.00 (4H, m), 3.04 (3H, s), 3.05-3.25 (2H, m), 3.50-3.65 (4H, m), 4.73 (2H, t, J=8.7 Hz), 6.45-6.70 (1H, br), 7.15-7.35 (4H, m), 7.70 (1H, s), 8.89 (1H, s).

### REFERENCE EXAMPLE 24

### 5-(2,3-Dihydro-1-benzofuran-5-yl)-5-oxopentanoic acid

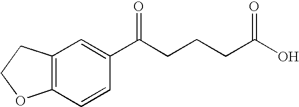

To a suspension (150 mL) of 2,3-dihydro-1-benzofuran (26.2 g, 221 mmol) and glutaric anhydride (25.3 g, 222 mmol) in dichloromethane, aluminum chloride (29.6 g, 222 mmol) was added by portions under ice-cooling. After stirring at 0° C. for 10 minutes, the reaction solution was poured into ice, 1 N hydrochloric acid (10 mL) was added thereto, and the mixture was extracted with ethyl acetate twice. The organic layer was washed with brine, and then dried over anhydrous magnesium sulfate. The organic layer was concentrated, and the precipitated solid was washed with a mixed solvent of ethanol and ethyl acetate to obtain the titled compound having a melting point of 131 to 133° C. as colorless crystals (14.1 g, 27%).

¹H NMR (300 MHz, CDCl₃) δ 2.07 (2H, quintet, J=7.2 Hz), 2.49 (2H, t, J=7.2 Hz), 3.01 (2H, t, J=7.2 Hz), 3.25 (2H, t, J=8.4 Hz), 4.66 (2H, t, J=8.4 Hz), 6.80 (1H, d, J=8.4 Hz), 7.80 (1H, dd, J=8.4, 1.8 Hz), 7.85 (1H, s).

Elemental analysis C₁₃H₁₄O₄,

Calcd: C, 66.66; H, 6.02.

Found: C, 66.48; H, 5.93.

### REFERENCE EXAMPLE 25

### Methyl 5-(2,3-dihydro-1-benzofuran-5-yl)-5-oxopentanoate

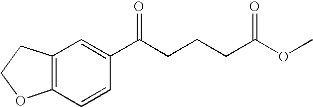

To a suspension of 5-(2,3-dihydro-1-benzofuran-5-yl)-5-oxopentanoic acid (17.2 g, 73.4 mmol) obtained in Reference Example 24 in methanol (500 mL), concentrated sulfuric acid (5 mL) was added portionwise. After refluxing under heating for 15 minutes, the mixture was cooled to room temperature, and was neutralized with saturated aqueous sodium hydrogen carbonate solution. Methanol was evaporated under reduced pressure, water was added to the residue, and the mixture was extracted with ethyl acetate. The organic layer was washed with brine, and dried over anhydrous magnesium sulfate, and then the organic layer was concentrated to obtain a white solid. The solid was crystallized from methanol-diethyl ether to obtain the titled compound having a melting point of 80 to 81° C. as colorless crystals (12.1 g). The mother liquor was filtered through a filtering column, and crystallized from methanol-diethyl ether to obtain 4.0 g of the titled compound. Total amount 16.1 g (88%).

¹H NMR (300 MHz, CDCl₃) δ 2.06 (2H, quintet, J=7.2 Hz), 2.44 (2H, t, J=7.2 Hz), 2.98 (2H, t, J=7.2 Hz), 3.25 (2H, t, J=8.7 Hz), 3.68 (3H, s), 4.66 (2H, t, J=8.7 Hz), 6.80 (1H, d, J=8.2 Hz), 7.80 (1H, dd, J=8.2, 1.8 Hz), 7.85 (1H, d, J=1.2 Hz).

Elemental analysis C₁₄H₁₆O₄,

Calcd: C, 67.73; H, 6.50.

Found: C, 67.77; H, 6.47.

### REFERENCE EXAMPLE 26

### Methyl 5-[7-(chlorosulfonyl)-2,3-dihydro-1-benzofuran-5-yl]-5-oxopentanoate

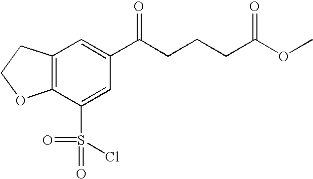

Methyl 5-(2,3-dihydro-1-benzofuran-5-yl)-5-oxopentanoate (12.0 g, 48.3 mmol) obtained in Reference Example 25 was added portionwise to a mixed solution of chlorosulfonic acid (60 mL) and thionyl chloride (6 mL) at room temperature. After stirring at room temperature for 2 hours, the reaction solution was portionwise poured into ice, and the mixture was extracted with ethyl acetate. The organic layer was washed with water, and then dried over anhydrous magnesium sulfate. The organic layer was concentrated, and the residue was purified by silica gel column chromatography and crystallized from diethyl ether-isopropyl ether to obtain the titled compound having a melting point of 78 to 79° C. as colorless crystals (8.05 g, 48%).

¹H NMR (300 MHz, CDCl₃) δ 2.07 (2H, quintet, J=7.2 Hz), 2.45 (2H, t, J=7.2 Hz), 3.03 (2H, t, J=7.2 Hz), 3.40 (2H, t, J=8.7 Hz), 3.70 (3H, s), 5.00 (2H, t, J=8.7 Hz), 8.16 (1H, s), 8.27 (1H, s).

Elemental analysis C₁₄H₁₅ClO₆S,

Calcd: C, 48.49; H, 4.36.

Found: C, 48.56; H, 4.41.

### REFERENCE EXAMPLE 27

### Methyl 5-[7-(aminosulfonyl)-2,3-dihydro-1-benzofuran-5-yl]-5-oxopentanoate

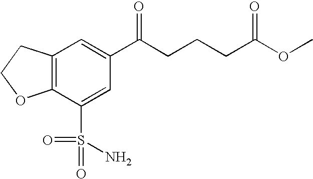

To a solution of methyl 5-[7-(chlorosulfonyl)-2,3-dihydro-1-benzofuran-5-yl]-5-oxopentanoate (7.82 g, 22.6 mmol) obtained in Reference Example 26 in THF (100 mL), a 28% ammonia solution (15.3 mL, 227 mmol) was added under ice-cooling. After stirring at room temperature for 15 minutes, ethyl acetate and 6 N hydrochloric acid (30 mL) were sequentially added, and then the aqueous layer was acidified. The mixture was extracted with ethyl acetate, and the organic layer was washed with brine and then dried over anhydrous magnesium sulfate. The organic layer was concentrated, and crystallized from methanol-THF-diethyl ether to obtain the titled compound having a melting point of 135 to 136° C. as colorless crystals (6.93 g, 94%).

¹H NMR (300 MHz, CDCl₃) δ 2.04 (2H, quintet, J=7.2 Hz), 2.43 (2H, t, J=7.2 Hz), 2.99 (2H, t, J=7.2 Hz), 3.35 (2H, t, J=8.7 Hz), 3.69 (3H, s), 4.91 (2H, t, J=8.7 Hz), 5.19 (2H, s), 8.03 (1H, s), 8.19 (1H, s)

Elemental analysis C₁₄H₁₇NO₆S,

Calcd: C, 51.37; H, 5.23; N, 4.28.

Found: C, 51.51; H, 5.31; N, 4.36.

### REFERENCE EXAMPLE 28

### 5-[7-(aminosulfonyl)-2,3-dihydro-1-benzofuran-5-yl]-5-oxopentanoic acid

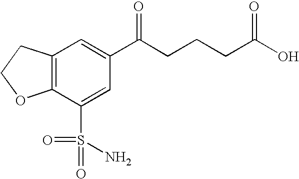

Methyl 5-[7-(aminosulfonyl)-2,3-dihydro-1-benzofuran-5-yl]-5-oxopentanoate (6.27 g, 20.5 mmol) obtained in Reference Example 27 was dissolved in a 1 N sodium hydroxide solution (300 mL), and the solution was stirred at room temperature for 3 hours. The solution was acidified by adding 6 N hydrochloric acid (51 mL), and concentrated under reduced pressure to the total amount of about 100 mL. The solution was extracted with a mixed solution of THF and ethyl acetate (1:1), and the organic layer was washed with brine, and then dried over anhydrous magnesium sulfate. The organic layer was concentrated, crystallized from THF-diethyl ether, and then further recrystallized from THF-diethyl ether to obtain the titled compound having a melting point of 207 to 209° C. as colorless crystals (6.16 g, 96%).

¹H NMR (300 MHz, DMSO-d₆) δ 1.82 (2H, quintet, J=7.2 Hz), 2.30 (2H, t, J=7.2 Hz), 3.01 (2H, t, J=7.2 Hz), 3.31 (2H, t, J=8.7 Hz), 4.81 (2H, t, J=8.7 Hz), 7.39 (2H, s), 8.05 (1H, s), 8.08 (1H, s), 12.08 (1H, s).

Elemental analysis C₁₃H₁₅NO₆S,

Calcd: C, 49.83; H, 4.83; N, 4.47.

Found: C, 49.89; H, 4.83; N, 4.30.

### REFERENCE EXAMPLE 29

### N-{5-[7-(Aminosulfonyl)-2,3-dihydro-1-benzofuran-5-yl]-5-oxopentyl}-2,2,2-trifluoro-N-{2-[2-(trifluoromethoxy)phenyl]ethyl}acetamide

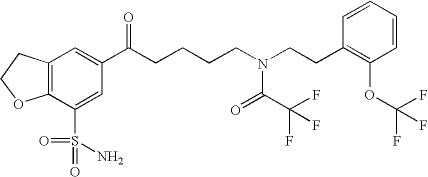

5-[5-({2-[2-(Trifluoromethoxy)phenyl]ethyl}amino)-pentanoyl]-2,3-dihydro-1-benzofuran-7-sulfonamide tosylate (5.00 g, 7.59 mmol) obtained in Example 6 was dissolved in a mixed solvent of THF (50 mL) and water (30 mL), and then a saturated aqueous potassium carbonate solution was added thereto to make the aqueous layer strongly basic. The mixture was extracted with a mixed solvent of ethyl acetate and THF (2:1) twice, and the organic layer was washed with a diluted potassium carbonate solution and brine. The organic layer was dried over anhydrous magnesium sulfate, and then the solvent was evaporated under reduced pressure to obtain a pale yellow oil. The oil was dissolved in THF (50 mL), trifluoroacetic anhydride (1.26 mL, 9.09 mmol) was added dropwise thereto at 0° C., and the mixture was stirred at room temperature for 3 hours. The reaction solution was concentrated under reduced pressure, water was added to the residue, and the mixture was extracted with ethyl acetate. The organic layer was washed with 1 N hydrochloric acid, a sodium hydrogen carbonate solution and brine, was dried over anhydrous magnesium sulfate, and then the solvent was evaporated under reduced pressure. The residue was purified by silica gel column chromatography, and crystallized from ethanol-diethyl ether to obtain the titled compound having a melting point of 108 to 110° C. as colorless crystals (2.99 g, 68%).

¹H NMR (300 MHz, CDCl₃) δ 1.55-1.80 (4H, m), 2.85-3.05 (4H, m), 3.20-3.35 (1H, m), 3.35 (2H, t, J=8.7 Hz), 3.40-3.65 (3H, m), 4.91 (2H, t, J=8.7 Hz), 5.14 (2H×½, s), 5.19 (2H×½, s), 7.20-7.35 (4H, m), 8.03 (1H, s), 8.19 (1H×½, s), 8.21 (1H×½, s)

### REFERENCE EXAMPLE 30

### N-{5-[7-(Aminosulfonyl)-2,3-dihydro-1-benzofuran-5-yl]-5-hydroxypentyl}-2,2,2-trifluoro-N-{2-[2-(trifluoromethoxy)phenyl]ethyl}acetamide

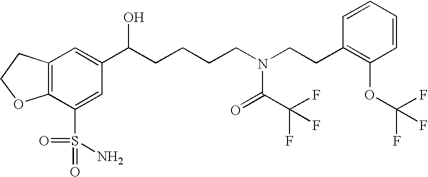

To a solution of N-{5-[7-(aminosulfonyl)-2,3-dihydro-1-benzofuran-5-yl]-5-oxopentyl}-2,2,2-trifluoro-N-{2-[2-(trifluoromethoxy)phenyl]ethyl}acetamide (2.50 g, 4.29 mmol) obtained in Reference Example 29 in a mixed solvent of methanol (30 mL) and THF (5 mL), sodium borohydride (162 mg) was added by portions under ice-cooling. After stirring at room temperature for 1 hour, the reaction solution was concentrated under reduced pressure, water was added to the residue, and the mixture was extracted with ethyl acetate. The organic layer was washed with brine, and dried over anhydrous magnesium sulfate, and then the solvent was evaporated under reduced pressure. The residue was purified by silica gel column chromatography to obtain the titled compound as a colorless oil (2.12 g, 85%).

¹H NMR (300 MHz, CDCl₃) δ 1.15-1.90 (6H, m), 2.10-2.35 (1H, m), 2.94 (2H, t, J=7.5 Hz), 3.15-3.30 (3H, m), 3.40 (1H, t, J=7.5 Hz), 3.50-3.60 (2H, m), 4.55-4.65 (1H, m), 4.80 (2H, t, J=8.7 Hz), 5.10-5.20 (2H, m), 7.20-7.35 (4H, m), 7.39 (1H, s), 7.50 (1H, m).

### REFERENCE EXAMPLE 31

### Preparation of 5-(1-{2-[2-(trifluoromethoxy)phenyl]ethyl}-1,4,5,6-tetrahydropyridin-2-yl)-2,3-dihydro-1-benzofuran-7-sulfonamide

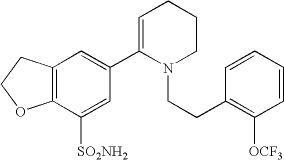

To a 300 mL four-neck flask equipped with a Dean Stark device, 5-(5-chloropentanoyl)-2,3-dihydro-1-benzofuran-7-sulfonamide (19.08 g, 60.0 mmol) and sodium iodide (8.98 g, 60.0 mmol) were poured, and the mixture was suspended in toluene (134 mL). The mixture was stirred under heating in an oily bath at 130° C., and reflux was started. {2-[2-(trifluoromethoxy)phenyl]ethyl}amine (14.78 g, 72.0 mmol) and N,N-diisopropylethylamine (7.76 g, 60.0 mmol) were sequentially added thereto. While separating off the produced water, the residue was stirred under heating for 1 hour and 20 minutes. The mixture was cooled to room temperature by water-cooling, and extracted with water and ethyl acetate (each 45 mL). The organic layer was sequentially washed with water, brine and water (each 45 mL). The organic layer was concentrated under reduced pressure and dried. Dried toluene was added thereto (50 mL×2), and further concentrated under reduced pressure to obtain 30.35 g of a brown oily titled compound.

¹H NMR (DMSO-d₆): δ 1.60-1.80(2H, m), 2.00-2.20(2H, m), 2.60-2.90(4H, m), 3.00-3.20(4H, m), 4.60-4.75(2H, m), 4.75-4.85(1H, m), 6.90-7.00(1H, m), 7.10-7.40(4H, m), 7.37-7.47(1H, m)

### EXAMPLE 1

### 6-[5-({2-[2-(Trifluoromethoxy)phenyl]ethyl}amino)-pentanoyl]indane-4-sulfonamide hydrochloride

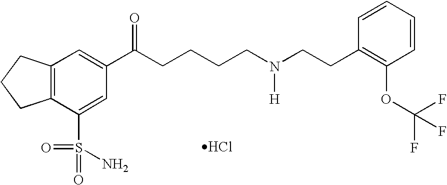

To a solution of tert-butyl 5-[7-(aminosulfonyl)-2,3-dihydro-1H-inden-5-yl]-5-oxopentyl{2-[2-(trifluoromethoxy)phenyl]ethyl}carbamate (570 mg, 1.03 mmol) obtained in Reference Example 5 in ethanol (10 mL), a 4 N hydrogen chloride-ethyl acetate solution (10 mL) was added, and the mixture was stirred at room temperature for 1 hour. The solvent was evaporated under reduced pressure, and the residue was crystallized from ethanol-ethyl acetate to obtain the titled compound having a melting point of 139 to 141° C. as colorless crystals (478 mg, 89%).

¹H NMR (300 MHz, DMSO-d₆) δ 1.60-1.80 (4H, m), 2.10 (2H, quintet, J=7.5 Hz), 2.90-3.15 (10H, m), 3.23 (2H, t, J=7.5 Hz), 7.35-7.55 (6H, m), 8.07 (1H, s), 8.18 (1H, s), 9.10-9.40 (2H, br).

Elemental analysis C₂₃H₂₇F₃N₂O₄S.HCl,

Calcd: C, 53.02; H, 5.42; N, 5.38.

Found: C, 52.93; H, 5.38; N, 5.27.

### EXAMPLE 2

### 6-(5-{[2-(2-Methoxyphenyl)ethyl]amino}pentanoyl)-indane-4-sulfonamide hydrochloride

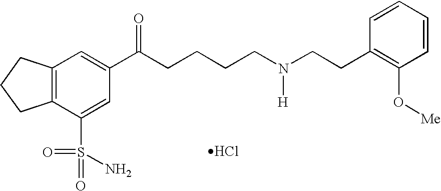

Using tert-butyl {5-[7-(aminosulfonyl)-2,3-dihydro-1H-inden-5-yl]-5-oxopentyl}[2-(2-methoxyphenyl)ethyl]carbamate (914 mg, 1.72 mmol) obtained in Reference Example 6, the same procedure as in Example 1 was carried out to obtain the titled compound having a melting point of 106 to 108° C. as colorless crystals (305 mg, 38%).

¹H NMR (300 MHz, DMSO-d₆) δ 1.60-1.80 (4H, m), 2.10 (2H, quintet, J=7.5 Hz), 2.90-3.10 (10H, m), 3.23 (2H, t, J=7.5 Hz), 3.80 (3H, s), 6.92 (1H, dt, J=7.5, 0.9 Hz), 7.00 (1H, dd, J=7.9, 0.9 Hz), 7.18 (1H, dd, J=7.5, 1.5 Hz), 7.26 (1H, dt, J=7.9, 1.5 Hz), 7.48 (2H, s), 8.07 (1H, s), 8.17 (1H, s), 8.80-9.00 (2H, br).

Elemental analysis C₂₃H₃₀N₂O₄S.HCl.0.5H₂O,

Calcd: C, 58.03; H, 6.78; N, 5.88.

Found: C, 57.95; H, 6.79; N, 5.89.

### EXAMPLE 3

### 6-(5-{[2-(2-Chlorophenyl)ethyl]amino}pentanoyl)indane-4-sulfonamide hydrochloride

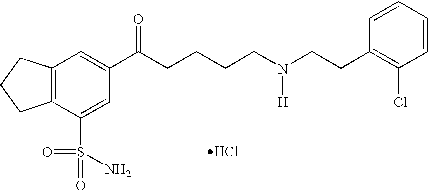

Using tert-butyl 5-[7-(aminosulfonyl)-2,3-dihydro-1H-inden-5-yl]-5-oxopentyl[2-(2-chlorophenyl)ethyl]carbamate (1.04 g, 1.95 mmol) obtained in Reference Example 7, the same procedure as in Example 1 was carried out to obtain the titled compound having a melting point of 167 to 169° C. as colorless crystals (599 mg, 65%).

¹H NMR (300 MHz, DMSO-d₆) δ 1.60-1.80 (4H, m), 2.10 (2H, quintet, J=7.5 Hz), 2.90-3.20 (10H, m), 3.23 (2H, t, J=7.5 Hz), 7.25-7.55 (6H, m), 8.07 (1H, s), 8.18 (1H, s), 9.10-9.30 (2H, br).

Elemental analysis C₂₂H₂₇ClN₂O₃S.HCl,

Calcd: C, 56.05; H, 5.99; N, 5.94.

Found: C, 55.76; H, 5.93; N, 5.92.

### EXAMPLE 4

### N-Methyl-6-[5-({2-[2-(trifluoromethoxy)phenyl]ethyl}-amino)pentanoyl]indane-4-sulfonamide hydrochloride

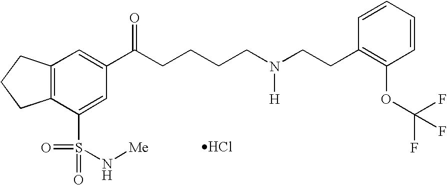

Using tert-butyl 5-{7-[(methylamino)sulfonyl]-2,3-dihydro-1H-inden-5-yl}-5-oxopentyl{2-[2-(trifluoromethoxy)-phenyl]ethyl}carbamate (570 mg, 0.95 mmol) obtained in Reference Example 8, the same procedure as in Example 1 was carried out to obtain the titled compound having a melting point of 143 to 145° C. as colorless crystals (303 mg, 60%).

¹H NMR (300 MHz, DMSO-d₆) δ 1.60-1.80 (4H, m), 2.10 (2H, quintet, J=7.5 Hz), 2.42 (3H, d, J=4.8 Hz), 2.90-3.10 (10H, m), 3.21 (2H, t, J=7.5 Hz), 7.35-7.50 (4H, m), 7.55-7.70 (1H, m), 8.11 (2H, s), 9.00-9.25 (2H, br).

Elemental analysis C₂₄H₂₉F₃N₂O₄S.HCl,

Calcd: C, 53.88; H, 5.65; N, 5.24.

Found: C, 53.71; H, 5.69; N, 5.17.

### EXAMPLE 5

### 5-[5-({2-[2-(Trifluoromethoxy)phenyl]ethyl}amino)-pentanoyl]-2,3-dihydro-1-benzofuran-7-sulfonamide hydrochloride

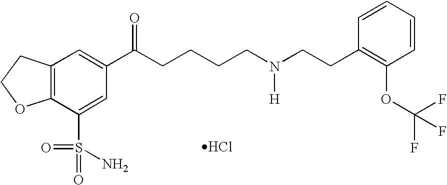

Using tert-butyl {5-[7-(aminosulfonyl)-2,3-dihydro-1-benzofuran-5-yl]-5-oxopentyl}{2-[2-(trifluoromethoxy)-phenyl]ethyl}carbamate (2.53 g, 4.31 mmol) obtained in Reference Example 9, the same procedure as in Example 1 was carried out to obtain the titled compound having a melting point of 135 to 137° C. as colorless crystals (1.36 g, 60%).

¹H NMR (300 MHz, DMSO-d₆) δ 1.60-1.80 (4H, m), 2.90-3.20 (6H, m), 3.25-3.45 (4H, m), 4.88 (2H, t, J=8.7 Hz), 7.35-7.55 (6H, m), 8.08 (1H, s), 8.11 (1H, s), 9.05-9.30 (2H, br).

Elemental analysis C₂₂H₂₅F₃N₂O₅S.HCl 0.5H₂O,

Calcd: C, 49.67; H, 5.12; N, 5.27.

Found: C, 49.86; H, 5.25; N, 5.39.

### EXAMPLE 6

### 5-[5-({2-[2-(Trifluoromethoxy)phenyl]ethyl}amino)-pentanoyl]-2,3-dihydro-1-benzofuran-7-sulfonamide tosylate

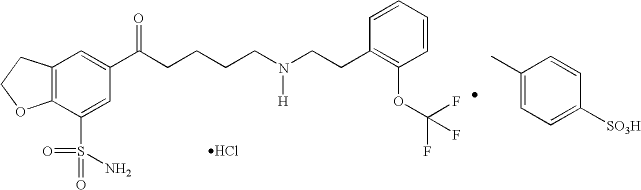

To a solution of tert-butyl {5-[7-(aminosulfonyl)-2,3-dihydro-1-benzofuran-5-yl]-5-oxopentyl}{2-[2-(trifluoromethoxy)phenyl]ethyl}carbamate (523 mg, 0.89 mmol) obtained in Reference Example 9 in ethanol (5 mL), a 4 N hydrogen chloride/ethyl acetate solution (10 mL) was added, and the mixture was stirred at room temperature for 3 hours. The mixture was concentrated under reduced pressure, and then the residue was dissolved in tetrahydrofuran (5 mL)/water (3 mL). To the solution, a saturated potassium carbonate solution (3 mL) was added, and the mixture was extracted with a mixed solvent of ethyl acetate and tetrahydrofuran (2:1) (20 mL, three times). The organic layer was washed with brine, dried over anhydrous magnesium sulfate, and then filtered through a filter paper. To the filtrate, a solution of p-toluene sulfonic acid monohydrate (168 mg, 0.88 mmol) in ethanol (5 mL) was added, and the mixture was concentrated under reduced pressure to obtain the titled compound having a melting point of 157 to 159° C. as colorless crystals (541 mg, 92%).

¹H NMR (300 MHz, DMSO-d₆) δ 1.60-1.75 (4H, m), 2.28 (3H, s), 2.90-3.20 (8H, m), 3.32 (2H, t, J=8.7 Hz), 4.83 (2H, t, J=8.7 Hz), 7.11 (2H, d, J=7.8 Hz), 7.35-7.55 (8H, m), 8.08 (1H, s), 8.10 (1H, s), 8.40-8.65 (2H, br).

Elemental analysis C₂₂H₂₅F₃N₂O₅S.C₇H₈O₃S,

Calcd: C, 52.88; H, 5.05; N, 4.25.

Found: C, 52.84; H, 4.85; N, 4.18.

Powder X-ray crystal diffraction: surface spacings (d values); about 25.4, about 12.8, about 11.2, about 8.56, about 6.42, about 5.32, about 5.13, about 4.44, and about 4.28 Angstroms.

### EXAMPLE 7

### 5-[5-({2-[2-(Trifluoromethoxy)phenyl]ethyl}amino)-pentanoyl]-2,3-dihydro-1-benzofuran-7-sulfonamide methanesulfonate

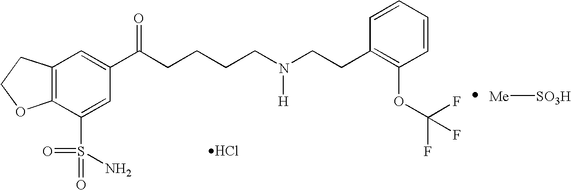

To 5-[5-({2-[2-(trifluoromethoxy)phenyl]ethyl}amino)-pentanoyl]-2,3-dihydro-1-benzofuran-7-sulfonamide hydrochloride (1.00 g, 1.91 mmol) obtained in Example 5, a 1 N sodium hydroxide solution (10 mL) was added to make basic the solution. The mixture was extracted with ethyl acetate twice. The organic layer was dried over anhydrous sodium sulfate and concentrated to obtain 5-[5-({2-[2-(trifluoromethoxy)phenyl]ethyl}amino)pentanoyl]-2,3-dihydro-1-benzofuran-7-sulfonamide (in the free base form) as a colorless oil (about 790 mg). The obtained product in the free base form was dissolved in ethanol (10 mL), and a solution of methane sulfonic acid (184 mg, 1.91 mmol) in ethanol (5 mL) was added thereto under ice-cooling. The solvent was evaporated under reduced pressure, and crystallized from ethanol to obtain the titled compound having a melting point of 153 to 155° C. as colorless crystals (780 mg, 79%).

¹H NMR (300 MHz, DMSO-d₆) δ 1.60-1.75 (4H, m), 2.33 (3H, s), 2.90-3.20 (8H, m), 3.32 (2H, t, J=8.7 Hz), 3.38 (2H, s), 4.82 (2H, t, J=8.7 Hz), 7.35-7.55 (4H, m), 8.07 (1H, s), 8.09 (1H, s), 8.45-8.65 (2H, br).

### EXAMPLE 8

### 5-[5-({2-[2-(Trifluoromethoxy)phenyl]ethyl}amino)-pentanoyl]-2,3-dihydro-1-benzofuran-7-sulfonamide maleate

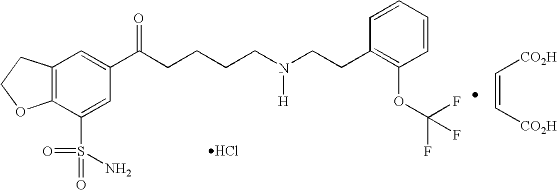

Using 5-[5-({2-[2-(trifluoromethoxy)phenyl]ethyl}-amino)pentanoyl]-2,3-dihydro-1-benzofuran-7-sulfonamide hydrochloride (1.00 g, 1.91 mmol) obtained in Example 5 and maleic acid (222 mg, 1.91 mmol), the same procedure as in Example 7 was carried out to obtain the titled compound having a melting point of 90 to 92° C. as colorless crystals (543 mg, 47%).

¹H NMR (300 MHz, DMSO-d₆) δ 1.60-1.75 (4H, m), 2.90-3.40 (10H, m), 3.32 (2H, t, J=8.7 Hz), 4.82 (2H, t, J=8.7 Hz), 6.03 (2H, s), 7.30-7.50 (5H, m), 8.07 (1H, s), 8.08 (1H, s), 8.35-8.60 (2H, br).

### EXAMPLE 9

### 5-[5-({2-[2-(Trifluoromethoxy)phenyl]ethyl}amino)-pentanoyl]-2,3-dihydro-1-benzofuran-7-sulfonamide fumarate

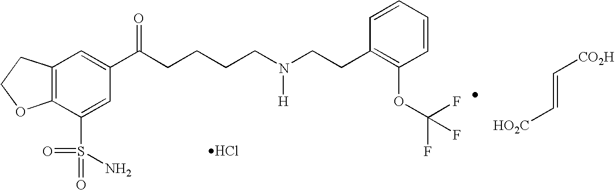

Using 5-[5-({2-[2-(trifluoromethoxy)phenyl]ethyl}-amino)pentanoyl]-2,3-dihydro-1-benzofuran-7-sulfonamide hydrochloride (1.00 g, 1.91 mmol) obtained in Example 5 and fumaric acid (222 mg, 1.91 mmol), the same procedure as in Example 7 was carried out to obtain the titled compound having a melting point of 143 to 145° C. as colorless crystals (840 mg, 73%).

¹H NMR (300 MHz, DMSO-d₆) δ 1.60-1.75 (4H, m), 2.70-3.40 (12H, m), 4.65 (2H, t, J=8.7 Hz), 4.78 (2H, s), 7.10-7.50 (7H, m), 8.05 (1H, s), 8.07 (1H, s).

### EXAMPLE 10

### 5-[5-({2-[2-(Trifluoromethoxy)phenyl]ethyl}amino)-pentanoyl]-2,3-dihydro-1-benzofuran-7-sulfonamide hydrobromide

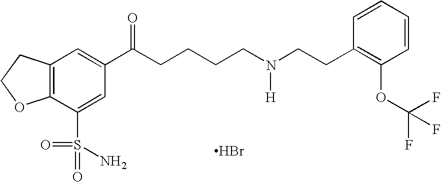

Using 5-[5-({2-[2-(trifluoromethoxy)phenyl]ethyl}-amino)pentanoyl]-2,3-dihydro-1-benzofuran-7-sulfonamide hydrochloride (1.00 g, 1.91 mmol) obtained in Example 5 and 48% hydrobromic acid (330 mg, 1.95 mmol), the same procedure as in Example 7 was carried out to obtain the titled compound having a melting point of 161 to 163° C. as colorless crystals (856 mg, 79%).

¹H NMR (300 MHz, DMSO-d₆) δ 1.55-1.75 (4H, m), 2.90-3.20 (8H, m), 3.32 (2H, t, J=8.7 Hz), 4.82 (2H, t, J=8.7 Hz), 7.30-7.50 (6H, m), 8.07 (1H, s), 8.09 (1H, s), 8.45-8.70 (2H, br).

### EXAMPLE 11

### 5-[6-({2-[2-(Trifluoromethoxy)phenyl]ethyl}amino)-hexanoyl]-2,3-dihydro-1-benzofuran-7-sulfonamide hydrochloride

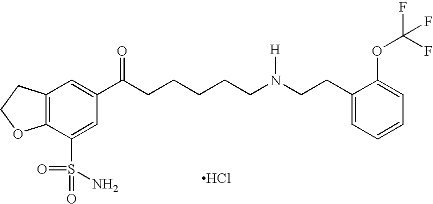

Using tert-butyl {6-[7-(aminosulfonyl)-2,3-dihydro-1-benzofuran-5-yl]-6-oxohexyl}{2-[2-(trifluoromethoxy)-phenyl]ethyl}carbamate (840 mg, 1.40 mmol) obtained in Reference Example 10, the same procedure as in Example 1 was carried out to obtain the titled compound having a melting point of 85 to 87° C. as colorless crystals (530 mg, 71%).

¹H NMR (300 MHz, DMSO-d₆) δ 1.39 (2H, quintet, J=7.2 Hz), 1.67 (4H, septet, J=7.2 Hz), 2.85-3.20 (6H, m), 2.99 (2H, t, J=7.2 Hz), 3.32 (2H, t, J=8.7 Hz), 4.82 (2H, t, J=8.7 Hz), 7.35-7.50 (6H, m), 8.07 (1H, d, J=1.5 Hz), 8.10 (1H, d, J=1.5 Hz), 9.10-9.30 (2H, br).

Elemental analysis C₂₃H₂₇F₃N₂O₅S.HCl.0.5H₂O,

Calcd: C, 50.59; H, 5.35; N, 5.13.

Found: C, 50.66; H, 5.43; N, 5.11.

### EXAMPLE 12

### 5-(6-{[2-(2-Methoxyphenyl)ethyl]amino}hexanoyl)-2,3-dihydro-1-benzofuran-7-sulfonamide hydrochloride

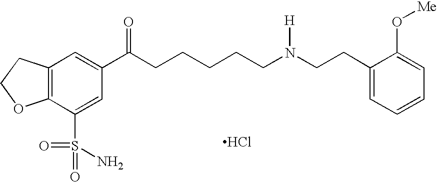

Using tert-butyl {6-[7-(aminosulfonyl)-2,3-dihydro-1-benzofuran-5-yl]-6-oxohexyl}[2-(2-methoxyphenyl)ethyl]-carbamate (662 mg, 1.21 mmol) obtained in Reference Example 11, the same procedure as in Example 1 was carried out to obtain the titled compound having a melting point of 92 to 94° C. as colorless crystals (564 mg, 97%).

¹H NMR (300 MHz, DMSO-d₆) δ 1.30-1.45 (2H, m), 1.55-1.75 (4H, m), 2.85-3.20 (8H, m), 3.32 (2H, t, J=8.7 Hz), 3.80 (3H, s), 4.82 (2H, t, J=8.7 Hz), 6.91 (1H, t, J=7.2 Hz), 7.00 (1H, d, J=8.1 Hz), 7.19 (1H, dd, J=7.2, 1.5 Hz), 7.26 (1H, dt, J=5.5, 1.5 Hz), 7.42 (2H, s), 8.07 (1H, s), 8.10 (1H, s), 8.95-9.10 (2H, br).

Elemental analysis C₂₃H₃₀N₂O₅S.HCl.0.5H₂O,

Calcd: C, 56.14; H, 6.56; N, 5.69.

Found: C, 56.49; H, 6.57; N, 5.73.

### EXAMPLE 13

### 5-(6-{[2-(2-Chlorophenyl)ethyl]amino}hexanoyl)-2,3-dihydro-1-benzofuran-7-sulfonamide hydrochloride

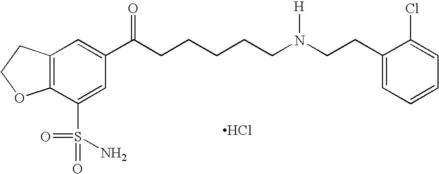

Using tert-butyl {6-[7-(aminosulfonyl)-2,3-dihydro-1-benzofuran-5-yl]-6-oxohexyl}[2-(2-chlorophenyl)-ethyl]carbamate (558 mg, 1.01 mmol) obtained in Reference Example 12, the same procedure as in Example 1 was carried out to obtain the titled compound having a melting point of 107 to 109° C. as colorless crystals (457 mg, 93%).

¹H NMR (300 MHz, DMSO-d₆) δ 1.30-1.45 (2H, m), 1.55-1.80 (4H, m), 2.80-3.20 (8H, m), 3.32 (2H, t, J=8.4 Hz), 4.82 (2H, t, J=8.4 Hz), 7.25-7.50 (6H, m), 8.07 (1H, s), 8.10 (1H, s), 9.10-9.40 (2H, br).

Elemental analysis C₂₂H₂₇ClN₂O₄S.HCl.0.5H₂O,

Calcd: C, 53.23; H, 5.89; N, 5.64.

Found: C, 53.37; H, 6.02; N, 5.56.

### EXAMPLE 14

### N-Methyl-5-[5-({2-[2-(trifluoromethoxy)phenyl]ethyl}-amino)pentanoyl]-2,3-dihydro-1-benzofuran-7-sulfonamide hydrochloride

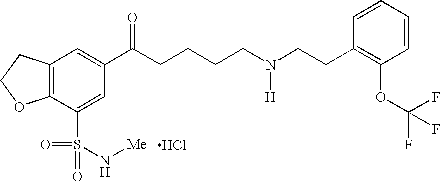

Using tert-butyl (5-{7-[(methylamino)sulfonyl]-2,3-dihydro-1-benzofuran-5-yl}-5-oxopentyl){2-[2-(trifluoromethoxy)phenyl]ethyl}carbamate (2.53 g, 4.31 mmol) obtained in Reference Example 13, the same procedure as in Example 1 was carried out to obtain the titled compound having a melting point of 135 to 137° C. as colorless crystals (1.36 g, 60%).

¹H NMR (300 MHz, DMSO-d₆) δ 1.60-1.80 (4H, m), 2.47 (3H, d, J=5.1 Hz), 2.90-3.15 (8H, m), 3.23 (2H, t, J=8.7 Hz), 4.82 (2H, t, J=8.7 Hz), 7.35-7.50 (5H, m), 8.07 (1H, s), 8.11 (1H, s), 9.10-9.30 (2H, br).

Elemental analysis C₂₃H₂₇F₃N₂O₅S.HCl,

Calcd: C, 51.44; H, 5.26; N, 5.22.

Found: C, 51.27; H, 5.27; N, 5.14.

### EXAMPLE 15

### N-Isopropyl-5-[5-({2-[2-(trifluoromethoxy)phenyl]-ethyl}amino)pentanoyl]-2,3-dihydro-1-benzofuran-7-sulfonamide hydrochloride

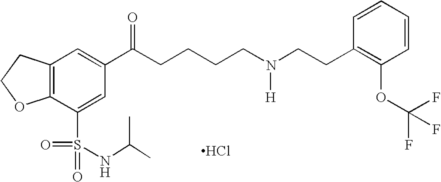

Using tert-butyl (5-{7-[(isopropylamino)sulfonyl]-2,3-dihydro-1-benzofuran-5-yl}-5-oxopentyl){2-[2-(trifluoromethoxy)phenyl]ethyl}carbamate obtained in Reference Example 14 (2.28 g, 3.62 mmol), the same procedure as in Example 1 was carried out to obtain the titled compound having a melting point of 173 to 175° C. as colorless crystals (1.61 g, 79%).

¹H NMR (300 MHz, DMSO-d₆) δ 0.98 (6H, d, J=6.6 Hz), 1.60-1.80 (4H, m), 2.90-3.20 (8H, m), 3.25-3.50 (3H, m), 4.82 (2H, t, J=8.7 Hz), 7.35-7.50 (4H, m), 7.60 (1H, d, J=7.8 Hz), 8.09 (2H, s), 9.05-9.25 (2H, br).

Elemental analysis C₂₅H₃₁F₃N₂O₅S.HCl,

Calcd: C, 53.14; H, 5.71; N, 4.96.

Found: C, 52.94; H, 5.70; N, 4.82.

### EXAMPLE 16

### 2-Methoxy-5-[5-({2-[2-(trifluoromethoxy)phenyl]-ethyl}amino)pentanoyl]benzenesulfonamide hydrochloride

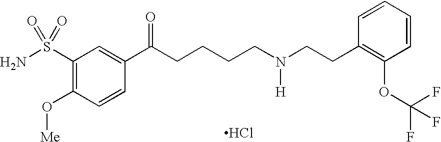

Using tert-butyl {5-[3-(aminosulfonyl)-4-methoxyphenyl]-5-oxopentyl}{2-[2-(trifluoromethoxy)-phenyl]ethyl}carbamate (674 mg, 1.17 mmol) obtained in Reference Example 15, the same procedure as in Example 1 was carried out to obtain the titled compound having a melting point of 169 to 171° C. as colorless crystals (490 mg, 82%).

¹H NMR (300 MHz, DMSO-d₆) δ 1.60-1.80 (4H, m), 2.90-3.20 (8H, m), 4.00 (3H, s), 7.25 (2H, s), 7.30-7.50 (5H, m), 8.22 (1H, dd, J=8.7, 2.4 Hz), 8.31 (1H, d, J=2.4 Hz), 9.10-9.25 (2H, br).

Elemental analysis C₂₁H₂₅F₃N₂O₅S.HCl,

Calcd: C, 49.36; H, 5.13; N, 5.48.

Found: C, 49.22; H, 5.20; N, 5.46.

### EXAMPLE 17

### N-Isopropyl-2-methoxy-5-[5-({2-[2-(trifluoromethoxy)-phenyl]ethyl}amino)pentanoyl]benzenesulfonamide hydrochloride

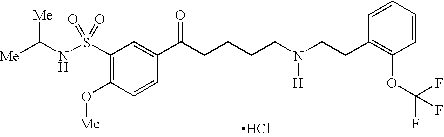

Using tert-butyl (5-{3-[(isopropylamino)sulfonyl]-4-methoxyphenyl}-5-oxopentyl){2-[2-(trifluoromethoxy)-phenyl]ethyl}carbamate (480 mg, 0.778 mmol) obtained in Reference Example 16, the same procedure as in Example 1 was carried out to obtain the titled compound having a melting point of 156 to 158° C. as colorless crystals (310 mg, 70%).

¹H NMR (300 MHz, DMSO-d₆) δ 0.957 (6H, d, J=6.6 Hz), 1.60-1.80 (4H, m), 2.90-3.40 (9H, m), 4.01 (3H, s), 7.30-7.50 (6H, m), 8.25 (1H, dd, J=8.7, 2.4 Hz), 8.52 (1H, d, J=2.4 Hz), 9.10-9.25 (2H, br).

Elemental analysis C₂₄H₃₁F₃N₂O₅S.HCl,

Calcd: C, 52.12; H, 5.83; N, 5.07.

Found: C, 51.90; H, 5.92; N, 5.03.

### EXAMPLE 18

### 7-[5-({2-[2-(Trifluoromethoxy)phenyl]ethyl}amino)-pentanoyl]-2,3-dihydro-1,4-benzodioxine-5-sulfonamide hydrochloride

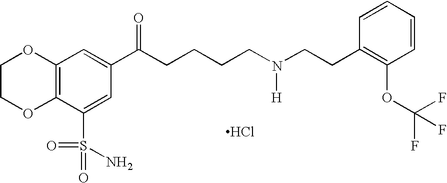

Using tert-butyl {5-[8-(aminosulfonyl)-2,3-dihydro-1,4-benzodioxin-6-yl]-5-oxopentyl}{2-[2-(trifluoromethoxy)phenyl]ethyl}carbamate (602 mg, 1.00 mmol) obtained in Reference Example 17, the same procedure as in Example 1 was carried out to obtain the titled compound having a melting point of 111 to 113° C. as colorless crystals (290 mg, 62%).

¹H NMR (300 MHz, DMSO-d₆) δ 1.60-1.80 (4H, m), 2.90-3.20 (8H, m), 4.30-4.40 (2H, m), 4.40-4.50 (2H, m), 7.30-7.50 (6H, m), 7.70 (1H, d, J=2.1 Hz), 7.90 (1H, d, J=2.1 Hz), 9.05-9.25 (2H, br).

Elemental analysis C₂₂H₂₅F₃N₂O₆S.HCl,

Calcd: C, 49.03; H, 4.86; N, 5.20.

Found: C, 48.70; H, 5.13; N, 4.93.

### EXAMPLE 19

### N-{5-[5-({2-[2-(Trifluoromethoxy)phenyl]ethyl}amino)pentanoyl]-2,3-dihydro-1-benzofuran-7-yl}acetamide hydrochloride

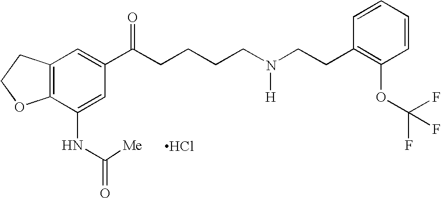

Using tert-butyl {5-[7-(acetylamino)-2,3-dihydro-1-benzofuran-5-yl]-5-oxopentyl}{2-[2-(trifluoromethoxy)-phenyl]ethyl}carbamate (1.18 g, 2.09 mmol) obtained in Reference Example 22, the same procedure as in Example 1 was carried out to obtain the titled compound having a melting point of 120 to 122° C. as colorless crystals (833 mg, 80%).

¹H NMR (300 MHz, DMSO-d₆) δ 1.60-1.80 (4H, m), 2.90-3.20 (11H, m), 3.32 (2H, t, J=8.7 Hz), 4.76 (2H, t, J=8.7 Hz), 7.30-7.55 (4H, m), 7.82 (1H, s), 7.83 (1H, s), 9.20-9.50 (2H, br), 9.60 (1H, s).

Elemental analysis C₂₄H₂₇F₃N₂O₄.HCl.2.5H₂O,

Calcd: C, 52.80; H, 6.09; N, 5.13.

Found: C, 52.54; H, 5.71; N, 5.48.

### EXAMPLE 20

### N-{5-[5-({2-[2-(Trifluoromethoxy)phenyl]ethyl}amino)-pentanoyl]-2,3-dihydro-1-benzofuran-7-yl}methanesulfonamide hydrochloride

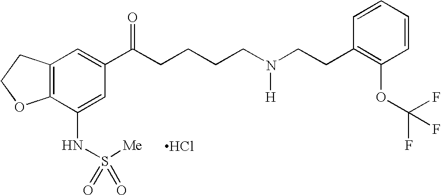

Using tert-butyl (5-{7-[(methylsulfonyl)amino]-2,3-dihydro-1-benzofuran-5-yl}-5-oxopentyl){2-[2-(trifluoromethoxy)phenyl]ethyl}carbamate (1.20 g, 2.00 mmol) obtained in Reference Example 23, the same procedure as in Example 1 was carried out to obtain the titled compound having a melting point of 112 to 114° C. as colorless crystals (420 mg, 39%).

¹H NMR (300 MHz, DMSO-d₆) δ 1.60-1.80 (4H, m), 2.90-3.20 (11H, m), 3.29 (2H, t, J=8.7 Hz), 4.71 (2H, t, J=8.7 Hz), 7.35-7.50 (4H, m), 7.69 (1H, s), 7.78 (1H, s), 9.05-9.25 (2H, br), 9.42 (1H, s).

Elemental analysis C₂₃H₂₇F₃N₂O₅S.HCl.H₂O,

Calcd: C, 49.77; H, 5.45; N, 5.05.

Found: C, 50.10; H, 5.32; N, 5.09.

### EXAMPLE 21

### 5-[1-Hydroxy-5-({2-[2-(trifluoromethoxy)phenyl]ethyl}-amino)pentyl]-2,3-dihydro-1-benzofuran-7-sulfonamide maleate

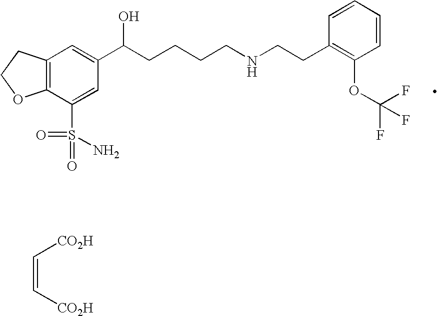

To a solution of N-{5-[7-(aminosulfonyl)-2,3-dihydro-1-benzofuran-5-yl]-5-hydroxypentyl}-2,2,2-trifluoro-N-{2-[2-(trifluoromethoxy)phenyl]ethyl}acetamide (2.12 g, 3.63 mmol) obtained in Reference Example 30 in methanol (30 mL), a saturated aqueous potassium carbonate solution (10 mL) and water (10 mL) were added, and the mixture was stirred at room temperature for 12 hours. The reaction solution was concentrated under reduced pressure, water was added to the residue, and the mixture was extracted with a mixed solution of ethyl acetate and THF (1:1) twice. The organic layer was washed with brine and dried over anhydrous sodium sulfate, and the solvent was evaporated under reduced pressure. The residue was purified by silica gel column chromatography to obtain the titled compound in the free base form as colorless amorphous powders (1.53 g, 86%).

¹H NMR (300 MHz, CDCl₃) δ 1.20-1.80 (6H, m), 2.50-2.65 (2H, m), 2.70-2.90 (4H, m), 3.00-4.30 (4H, br), 3.26 (2H, t, J=9.0 Hz), 4.55-4.60 (1H, m), 4.78 (2H, t, J=9.0 Hz), 7.20-7.35 (4H, m), 7.41 (1H, s), 7.50 (1H, s).

A solution of the obtained product in the free base form (600 mg, 1.23 mmol) in ethanol (10 mL) was cooled with crushed ice-sodium chloride mixture (to about −20° C.), and a solution of maleic acid (143 mg, 1.23 mmol) in ethanol (10 mL) which had been to 0° C. or lower was added dropwise thereto under stirring. The obtained solution was concentrated under reduced pressure at room temperature or lower to obtain the titled compound having a melting point of 61 to 63° C. as colorless crystals (551 mg, 70%).

¹H NMR (300 MHz, DMSO-d₆) δ 1.20-1.80 (6H, m), 2.80-3.20 (7H, m), 3.23 (2H, t, J=8.7 Hz), 4.50 (1H, s), 4.68 (2H, t, J=8.7 Hz), 5.27 (1H, s), 6.03 (2H, s), 7.15 (2H, s), 7.30-7.55 (6H, m), 8.30-8.70 (2H, br).

Elemental analysis C₂₂H₂₇F₃N₂O₅S.C₄H₄O₄.2H₂O,

Calcd: C, 48.75; H, 5.51; N, 4.37.

Found: C, 48.74; H, 5.50; N, 4.29.

### EXAMPLE 22

### Preparation of 5-[5-({2-[2-(Trifluoromethoxy)phenyl]-ethyl}amino)pentanoyl]-2,3-dihydro-1-benzofuran-7-sulfonamide tosylate

### (1) Preparation of 5-chloro-1-(2,3-dihydro-1-benzofuran-5-yl)pentan-1-one

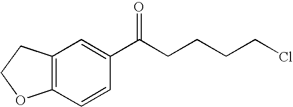

2,3-dihydrobenzofuran (35.6 g, 0.296 mol) and toluene (160 mL) were mixed, and the mixture was stirred. Anhydrous aluminum chloride (III) (39.5 g, 0.296 mol, 1.00 equivalent) was added thereto at 25° C. or lower. The mixture was stirred at 20 to 30° C. for 30 minutes. 5-Chloropentanoyl chloride (48.3 g, 0.312 mol, 1.05 equivalents) was added dropwise at −5 to 5° C., and the mixture was flushed with toluene (20 mL). The mixture was stirred at −5 to 5° C. for 1 hour. The reaction solution was added dropwise to ice-water (180 mL) at 25° C. or less. The organic layer was separated, and washed with water (180 mL) twice. The residue was concentrated under reduced pressure to about 110 mL. The residue was stirred at 20 to 30° C. for 30 minutes. n-Heptane (214 mL) was added dropwise thereto for 1 hour. After cooling, the mixture was stirred at 0 to 10° C. for 30 minutes. After taking out crystals, the crystals were dried in vacuo at about 50° C., to obtain the titled compound (65.6 g, yield 92.8%).

¹H NMR (CDCl₃): δ 1.86-1.88 (4H, br), 2.94 (2H, t, J=6.7 Hz), 3.25 (2H, t, J=8.7 Hz), 3.56-3.60 (2H, m), 4.66 (2H, t, J=8.8 Hz), 6.80 (1H, d, J=8.4 Hz), 7.80 (1H, d, J=8.4 Hz), 7.85 (1H, s).

### (2) Preparation of 5-(5-chloropentanoyl)-2,3-dihydro-1-benzofuran-7-sulfonamide

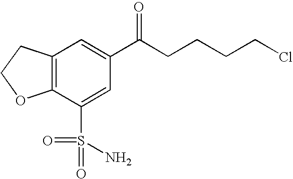

Thionyl chloride (18.7 g, 0.157 mol, 1.00 equivalent) and chlorosulfonic acid (73.0 g, 0.627 mol, 4.00 equivalents) were mixed, and the mixture was stirred. 5-Chloro-1-(2,3-dihydro-1-benzofuran-5-yl)-1-pentane (18.5 g, 77.5 mmol) was added thereto portionwise at 10° C. or lower. The mixture was stirred at 35 to 45° C. for 1.5 hours. The reaction solution was added dropwise to a mixed solution of ice-water (187 mL) and 4-methyl-2-pentanone (187 mL) at 15° C. or lower. The organic layer was separated at 20 to 30° C., and washed with brine (94 mL) four times. A 10% ammonia solution (112 mL) was added dropwise at 10° C. or lower. The mixture was stirred at 20 to 30° C. for 1 hour. Heptane (750 mL) was added dropwise thereto. After cooling, the mixture was stirred at 0 to 10° C. for 30 minutes. After taking out crystals, the crystals were dried in vacuo at about 50° C., to obtain the titled compound (22.3 g, yield 90.5%).

¹H NMR (DMSO-d₆): δ 1.70-1.83 (4H, br), 3.01 (2H, t, J=6.8 Hz), 3.31 (2H, t, J=9.4 Hz), 3.68 (2H, t, J=6.3 Hz), 4.81 (2H, t, J=8.8 Hz), 7.37 (2H, s), 8.08 (2H, br).

### (3) Preparation of 5-[5-({2-[2-(trifluoromethoxy)-phenyl]ethyl}amino)pentanoyl]-2,3-dihydro-1-benzofuran-7-sulfonamide tosylate

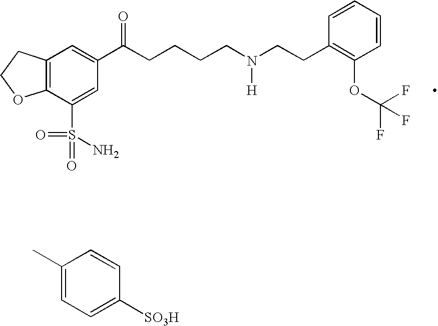

Under nitrogen atmosphere, 5-(5-chloropentanoyl)-2,3-dihydro-1-benzofuran-7-sulfonamide (31.8 g, 0.100 mol), {2-[2-(trifluoromethoxy)phenyl]ethyl}amine (32.8 g, 0.160 mol, 1.60 equivalents), sodium iodide (15.0 g, 0.100 mol, 1.00 equivalent), sodium carbonate (12.7 g, 0.120 mol, 1.20 equivalents), and n-propyl acetate (220 mL) were mixed, and the mixture was stirred. The refluxed solution was subject to water separation, while the solution was refluxed under heating for 3 hours. The solution was cooled to 40° C. or lower, and a 1 M aqueous sodium thiosulfate solution (100 mL) was added thereto. The organic layer was separated, and washed with water (100 mL) twice. The separated organic layer was added to a solution of tosylate monohydrate (28.5 g, 0.150 mol, 1.50 equivalents) in ethyl acetate (220 mL) at 50 to 60° C. The seed crystals (0.30 g) were added thereto, and the mixture was stirred at 50 to 60° C. for 1 hour, and then slowly cooled to 20° C. The mixture was left to stand at room temperature overnight. After cooling, the mixture was stirred at 0 to 10° C. for 1.5 hours. After taking out crystals, the crystals were dried in vacuo at about 50° C. to obtain the titled compound in crude crystals (55.3 g, 84.0 mmol, yield 84.0%). The crude crystals (30.0 g, 45.5 mmol), water (4.5 mL), and acetone (90 mL) were mixed, and the mixture was dissolved under heating at 50 to 60° C. To the mixture, ethyl acetate (90 mL) was added dropwise at 50 to 60° C. The seed crystals (0.15 g) were added thereto, and the mixture was stirred at 50 to 60° C. for 30 minutes. To the obtained suspension, ethyl acetate (240 mL) was added dropwise at 50 to 60° C. After cooling, the mixture was stirred at 0 to 10° C. for 1 hour. After taking out crystals, the crystals were dried in vacuo at about 50° C. to obtain the titled compound (28.6 g, 43.4 mmol, yield 95.4%).

¹H NMR (DMSO-d₆) δ 1.67 (4H, brs), 2.28 (3H, s), 2.97-3.33 (10H, m), 4.82 (2H, t, J=8.8 Hz), 7.11 (2H, d, J=8.0 Hz), 7.37-7.50 (8H, m), 8.10 (2H, d, J=7.1 Hz), 8.50 (2H, brs).

### EXPERIMENTAL EXAMPLE 1

a) Measurement of Acetylcholinesterase-Inhibitory Action

Acetylcholinesterase-inhibitory action of the Example Compounds was measured using acetylcholinesterase from human erythrocyte origin according to the acetylthiocholine method (Ellman method).

Acetylcholinesterase from human erythrocyte origin (Sigma Chemical Co.) was dissolved in distilled water at a concentration of 0.2 IU/mL to give an enzyme authentic sample. To a 96-well microplate 20 μL of drug solution, 30 μL of 80 mM Tris-HCl (pH 7.4), 50 μL of enzyme authentic sample and 50 μL of 5 mM 5,5-dithio-bis(2-nitrobenzoic acid) (Sigma Chemical Co.) were added, and the plate was shaken for 10 seconds. Then, 50 μL of acetylthiocholine iodide (Sigma Chemical Co.) was added, and the plate was again shaken. Immediately after shaking, absorbance at 414 nM was measured at intervals of 30 seconds for 10 minutes. The enzyme activity was determined according to the following formula:
R=5.74×10⁻⁷×DELTA A
wherein R indicates an enzyme activity (mol), and DELTA A shows change in absorbance at 414 nM.

The experiment was repeated at least 3 times for each compound to obtain 50% inhibitory concentration (IC₅₀). Moreover, in the same manner as mentioned above, acetylcholinesterase-inhibiting activity of distigmine was measured.

b) Measurement of α₁ A Receptor Binding Inhibitory Activity

The genetic engineering procedures as described below were based on the methods described in the textbook (Maniatis, et al., Molecular Cloning, Cold Spring Harbor Laboratory, 1989) or the methods described in the protocols attached to the reagents.

(i) Preparation of Expression Plasmid of Human Adrenaline α1 A Receptor

An adrenaline α1 A receptor gene was cloned from human liver cDNA by means of a PCR method. PCR reaction was performed in a Gene Amp PCR System 9700 (Applied Biosystems) with 200 ng of a human liver cDNA library (Takara Shuzo Co., Ltd.) as a template, by adding 50 pmol of each of the primer sets shown below which were prepared with referring to the base sequence of the adrenaline α1 A receptor gene reported by Hirasawa A. et al. (Biochem. Biophys. Res. Commun., 195, 902-909 (1993)): 5′-CCGAATTCGGCTGGGACCATGGTGTTTCTC-3′ [SEQ ID NO: 1], and 5′-CTGTCGACCTTTCCTGTCCTAGACTTCCTC-3′ [SEQ ID NO: 2], and using a TaKaRa Pyrobest DNA Polymerase (Takara Shuzo Co., Ltd.) (reaction condition: 45 cycles of 94° C. for 15 seconds and 68° C. for 3 minutes and 30 seconds).

Thus obtained PCR fragments were digested with a restriction enzyme Eco RI (Takara Shuzo Co., Ltd.) and Sal I (Takara Shuzo Co., Ltd.), and then subjected to electrophoresis on agarose gel to recover DNA fragments. The DNA fragments and the animal cell expression plasmid pMSRαneo digested with Eco RI and Sal I were mixed, and ligated to a DNA Ligation Kit Ver. 2 (Takara Shuzo Co., Ltd.), and then transformed into an E. Coli JM109 competent cell to obtain a plasmid pMSRaneo-Adre α1 A.

(ii) Introduction of Human Adrenaline α1 A Receptor Expression Plasmid to CHO—K1 Cell and Preparation of Membrane Fraction

CHO—K1 cells were grown in a cell culture flask of 150 cm² (Corning Coaster) using Ham's F12 Medium (Invitrogen) containing 10% fetal bovine serum (Trace Scientific), and scraped by treating with 0.5 g/L tripsin-0.2 g/L EDTA (Invitrogen). The cells were washed with D-PBS (−) (Invitrogen), centrifuged (1000 rpm, 5 minutes). Then, using a Gene Pulser II (BioRad), DNA was transfected into the cell under the following conditions. 1×10⁷ cells and 10 μg of pMSRαneo-Adreα1A were suspended in 700 μl of D-PBS (−), and added into a 0.4 cm gap cuvette (BioRad). DNA was transfected into the cells by electroporation using gene pulser II at 0.25 kV voltage and 960 μF capacitance. Stable transfectants were selected in the medium (Ham's F-12 medium (Invitrogen) supplemented with 10% FCS (Trace) and 500 μg/mL G418 (Invitrogen))

Stable transfectants were cultured to a semi-confluent state in a 150 cm² cell culture flask, and a cell membrane fraction was prepared in the following manner.

The cells in the semi-confluent state were scraped by treating with D-PBS (−) containing 0.02% EDTA, and centrifuged to obtain a cell pellet. The cell pellet was suspended in a buffer for preparation of membrane (10 mmol/L NaHCO₃ pH 7.4 with Protease Inhibitor Cocktails (Roche)) and the cells were homogenized using a Polytron homogenizer (Kinematica AG, Model PT-3100) at 20000 rpm for 20 seconds three times. The disrupted cells were centrifuged at 2000 rpm for 10 minutes to obtain the supernatant containing a membrane fraction. Further, the supernatant was centrifuged using an ultracentrifuge (Model L8-70M, Rotor 70Ti, Beckman) at 30000 rpm for 1 hour to obtain a precipitant containing a membrane fraction. Thus obtained membrane fraction of each clone was provided to the following binding test.

The membrane fraction (10 μg/well) and [³H]-Prazosin (2.5 nM, Perkin Elmer Life Sciences) diluted with binding assay buffer (50 mM Tris-HCl, 10 mM MgCl₂, 0.5% BSA with protease inhibitor cocktails, pH7.5) were added to a 96-well microplate and incubated at room temperature for 1 hour. For measurement of non-specific binding, Phentolamine (Sigma) was added to 10 μM. The reaction solution was filtered, and through Unifilter GF/C membrane filter plate (Perkin Elmer Life Sciences) by using Cell Harvester (Perkin Elmer Life Sciences). The membrane was washed three times with ice-cold 50 mM Tris buffer (pH 7.5). After drying the filter, 20 μL of Microscinti-O (Perkin Elmer Life Sciences) was added to each well and the membrane-associated radioactivity was measured with a top count (Perkin Elmer Life Sciences). A membrane fraction for evaluation of the compounds to be described below was prepared from transfectant, which showed the highest S/B value (entirely binding radioactivity/non-specific binding radioactivity) in the above-described manner.

(iii) Evaluation of Example Compound

The membrane fraction (10 μg/well), a compound and [³H]-Prazosin (2.5 nM, Perkin Elmer Life Sciences) diluted with binding assay buffer were added to a 96-well microplate and incubated at room temperature for 1 hour. For measurement of non-specific binding, further Phentolamine (Sigma) was added to 10 μM. The reaction solution was filtrated and transported to a Unifilter GF/C membrane filter plate (Perkin Elmer Life Sciences) by using Cell Harvester (Perkin Elmer Life Sciences). The membrane was washed three times with ice-cold 50 mM Tris buffer (pH 7.5). After drying the filter, 20 μL of Microscinti-O (Perkin Elmer Life Sciences) was added to each well and the membrane-associated radioactivity was measured with a top count (Perkin Elmer Life Sciences)

IC₅₀ (concentration of the compounds needed to inhibit the binding of [³H]-Prazosin to the membrane fraction by 50%) was calculated with SAS 8.2 software. IC₅₀ of urapidil (hydrochloride) which was a known α1 receptor antagonistic agent was determined in the same manner.

The results of the above methods a) and b) are presented in the following table.

TABLE 1

Compound

| | AChE: IC₅₀ (μM) | α1A: IC₅₀ (μM) |
|---|---|---|
| Example 6 | 0.080 | 0.019 |
| Distigmine | 0.27 | — |
| Urapidil | — | 0.47 |

As shown above, it was clear that compound (I) of the present invention has an excellent acetylcholinesterase inhibitory action, in combination with an excellent α₁ receptor antagonistic action.

### PREPARATION EXAMPLE 1

| | |
|---|---|
| (1) Compound of Example 6 | 1 g |
| (2) Lactose | 197 g |
| (3) Corn starch | 50 g |
| (4) Magnesium stearate | 2 g |

(1), (2) and corn starch (20 g) were mixed, and the mixture was granulated with a paste made from corn starch (15 g) and 25 mL of water, and corn starch (15 g) and (4) were added thereto. Thus obtained mixture was compressed using compression tableting device to prepare 2000 tablets, each tablet having a diameter of 3 mm and containing 0.5 mg of the compound of Example 6.

### PREPARATION EXAMPLE 2

| | |
|---|---|
| (1) Compound of Example 6 | 2 g |
| (2) Lactose | 197 g |
| (3) Corn starch | 50 g |
| (4) Magnesium stearate | 2 g |

In the same manner as in Preparation Example 1, 2000 tablets were prepared, each tablet having a diameter of 3 mm and containing 1.0 mg of the compound of Example 6.

### PREPARATION EXAMPLE 3

| | |
|---|---|
| (1) Compound of Example 6 | 5.0 mg |
| (2) Lactose | 60.0 mg |
| (3) Corn starch | 35.0 mg |
| (4) Gelatin | 3.0 mg |
| (4) Magnesium stearate | 2.0 mg |

A mixture of (1), (2) and (3) was granulated through a 1 mm-mesh sieve using 0.03 mL of a 10% by weight aqueous solution of gelatin (3.0 g as gelatin), and then dried at 40° C. and sieved. The obtained granules were mixed with (5) and compressed. The obtained core tablets were sugar-coated with an aqueous suspension of sucrose, titanium dioxide, talc and gum Arabic. The thus-coated tablets were glazed with bees wax to obtain a coated tablet.

### PREPARATION EXAMPLE 4

Preparations were prepared according to the prescription shown in Tables 2 and 3. That is, for example, for a 0.5 mg tablet, the compound of Example 6 (152.3 g), lactose (3956 g) and corn starch (450.0 g) were homogeneously mixed, a solution having hydroxypropylmethylcellulose (135.0 g) dissolved therein was sprayed for granulation and then dried in the machine. The obtained granules were crushed and screened with a 1.5 mmφ punching screen using a Power Mill grinder to obtain screened granules. Further, lactose (4109 g) and corn starch (450.0 g) were homogeneously mixed in a fluidized bed granulator, a solution having hydroxypropylmethylcellulose (135.0 g) dissolved therein was sprayed for granulation and then dried in the machine. The obtained granules were crushed and screened with a 1.5 mmφ punching screen using a Power Mill grinder to obtain placebo granules. The screened powder (938.7 g) and the placebo (3755 g) were mixed, croscarmellose calcium (225.0 g) and magnesium stearate (31.50 g) were added thereto, and the mixture was mixed with a Tumbler Mixer to obtain mixed granules. The obtained mixed granules were tableted to a tablet weighing 110 mg with a tableting machine using a 6.5 mmφ hammer. To the obtained tablet, a film coating solution containing hydroxypropylmethylcellulose (160.6 g), titanium oxide (18.00 g), yellow iron sesquioxide (0.540 g) and red iron sesquioxide (0.900 g) were sprayed using a pan type coating equipment to obtain a film-coated tablet. At this time, the condition was controlled so that the product temperature was adjusted to 40° C. to 50° C.

### PREPARATION EXAMPLE 5

Preparations were prepared according to the prescription shown in Table 2. That is, for example, for a 2.5 mg tablet, the compound of Example 6 (152.3 g), lactose (3956 g) and corn starch (450.0 g) were homogeneously mixed, a solution having hydroxypropylmethylcellulose (135.0 g) dissolved therein was sprayed for granulation and then dried in the machine. The obtained granules were crushed and screened with a 1.5 mmφ punching screen using a Power Mill grinder to obtain screened granules. Further, 4693 g of the obtained granules were taken, and croscarmellose calcium (225.0 g) and magnesium stearate (31.50 g) were added thereto, and the mixture was mixed with a Tumbler Mixer to obtain mixed granules. The obtained mixed granules were tableted to a tablet weighing 110 mg with a tableting machine using a 6.5 mmφ hammer. To the obtained tablet, a film coating solution containing hydroxypropylmethylcellulose (160.6 g), titanium oxide (18.00 g), yellow iron sesquioxide (0.540 g) and red iron sesquioxide (0.900 g) were sprayed using a pan type coating equipment to obtain a film-coated tablet. At this time, the condition was controlled so that the product temperature was adjusted to 40° C. to 50° C. Similarly, a 10 mg of tablet was prepared by controlling the contents of the compound of Example 6 and lactose in the granules of the compound of Example 6.

TABLE 2

| | 0.5 mg | | 2.5 mg | | 10 mg | |
|---|---|---|---|---|---|---|
| | tablet** | | tablet | | tablet | |
| Compound of Example 6 | 0.677 | mg | 3.385 | mg | 13.54 | mg |
| Lactose | 90.623 | mg | 87.915 | mg | 77.76 | mg |
| Corn starch | 10 | mg | 10 | mg | 10 | mg |
| Hydroxypropylmethylcellulose | 3 | mg | 3 | mg | 3 | mg |
| Croscarmellose calcium | 5 | mg | 5 | mg | 5 | mg |
| Magnesium stearate | 0.7 | mg | 0.7 | mg | 0.7 | mg |
| Hydroxypropylmethylcellulose | 3.568 | mg | 3.568 | mg | 3.568 | mg |
| Titanium oxide | 0.4 | mg | 0.4 | mg | 0.4 | mg |
| Yellow iron sesquioxide | 0.012 | mg | 0.012 | mg | 0.012 | mg |
| Red iron sesquioxide | 0.02 | mg | 0.02 | mg | 0.02 | mg |
| Total | 114 | mg | 114 | mg | 114 | mg |

*: The compound of Example 6 is tosylate (M.W. 658.71), and the amount thereof to be injected is converted to that of a free product (M.W. 486.51), based on the converted value (1.354).
**For 0.5 mg of a tablet, a method was carried out, in which the following sized granules and the placebo granules were separately subject to granulation and sizing, and then mixed.

TABLE 3

| | |
|---|---|
| | Placebo |
| Sized granules | granules |

| Compound of Example 6 | 0.677 | mg | — | |
|---|---|---|---|---|
| Lactose | 17.583 | mg | 73.04 | mg |
| Corn starch | 2 | mg | 8 | mg |
| Hydroxypropylmethylcellulose | 0.6 | mg | 2.4 | mg |
| Total | 20.86 | mg | 83.44 | mg |

### INDUSTRIAL APPLICABILITY

The compound of the present invention has a combined effect of an acetylcholinesterase inhibitory action and an α₁ receptor antagonistic action, and a high effect of improving the excretion function of the urinary bladder (effect of improving the flow rate of urine and the urination efficiency) as well as not affecting the urination pressure and the blood pressure, thus it being useful as an agent for preventing and/or treating lower urinary tract symptoms.

1. A compound represented by the formula: 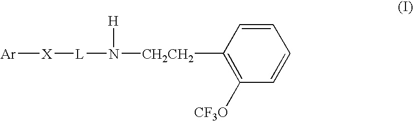
wherein Ar represents a group represented by the formula: 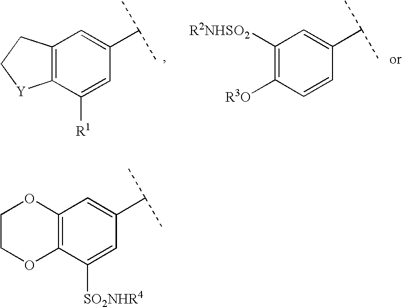
(wherein Y represents methylene or an oxygen atom, R¹ represents aminosulfonyl, C₁₋₆ alkyl-aminosulfonyl, C₁₋₆ alkyl-carbonylamino or C₁₋₆ alkyl-sulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group; and L represents an optionally substituted C₄₋₅ alkylene group, or a salt thereof.

2. 6-[5-({2-[2-(Trifluoromethoxy)phenyl]ethyl}-amino)pentanoyl]indane-4-sulfonamide or a salt thereof.

3. 5-[5-({2-[2-(Trifluoromethoxy)phenyl]ethyl}-amino)pentanoyl]-2,3-dihydro-1-benzofuran-7-sulfonamide or a salt thereof.

4. N-{5-[5-({2-[2-(Trifluoromethoxy)phenyl]ethyl}-amino)pentanoyl]-2,3-dihydro-1-benzofuran-7-yl}-methanesulfonamide or a salt thereof.

5. 5-[1-Hydroxy-5-({2-[2-(trifluoromethoxy)phenyl]-ethyl}amino)pentyl]-2,3-dihydro-1-benzofuran-7-sulfonamide or a salt thereof.

6. Crystals of a salt of 5-[5-({2-[2-(trifluoromethoxy)phenyl]ethyl}amino)pentanoyl]-2,3-dihydro-1-benzofuran-7-sulfonamide having a melting point of 90° C. or higher.

7. Crystals of 5-[5-({2-[2-(trifluoromethoxy)phenyl]-ethyl}amino)pentanoyl]-2,3-dihydro-1-benzofuran-7-sulfonamide p-toluene sulfonate.

8. Crystals of 5-[5-({2-[2-(trifluoromethoxy)-phenyl]ethyl}amino)pentanoyl]-2,3-dihydro-1-benzofuran-7-sulfonamide p-toluene sulfonate having a melting point of about 153° C. to about 163° C.

9. A method for preparing 5-[5-({2-[2-(trifluoromethoxy)phenyl]ethyl}amino)pentanoyl]-2,3-dihydro-1-benzofuran-7-sulfonamide or a salt thereof, comprising reacting a compound represented by the formula: 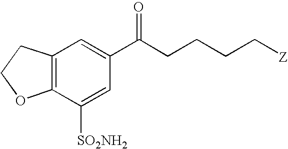
wherein Z represents a leaving group,
or a salt thereof with 2-[2-(trifluoromethoxy)-phenyl]ethylamine or a salt thereof under dehydration conditions and hydrolyzing the resulting product.

10. A pharmaceutical composition comprising a compound represented by the formula: 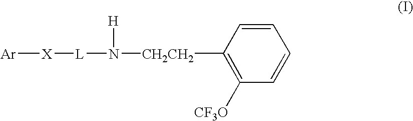
wherein Ar represents a group represented by the formula: 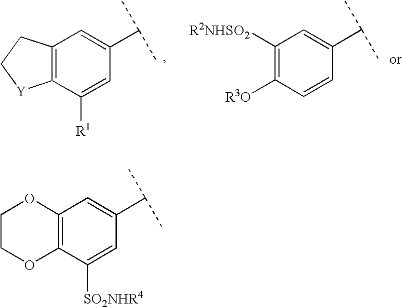
(wherein Y represents methylene or an oxygen atom, R¹ represents aminosulfonyl, C₁₋₆ alkyl-aminosulfonyl, C₁₋₆ alkyl-carbonylamino or C₁₋₆ alkyl-sulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group; and L represents an optionally substituted C₄₋₅ alkylene group, or a salt thereof, or a prodrug thereof.

11. The pharmaceutical composition according to claim 10, having a combined effect of an acetylcholinesterase inhibitory action and an α₁ receptor antagonistic action.

12. The pharmaceutical composition according to claim 10, which is an agent for preventing and/or treating lower urinary tract symptoms.

13. The pharmaceutical composition according to claim 10, which is an agent for preventing and/or treating lower urinary tract symptoms accompanied by benign prostatic hyperplasia.

14. The pharmaceutical composition according to claim 10, which is an agent for preventing and/or treating lower urinary tract symptoms by bladder underactivity.

15. A method for preventing and/or treating lower urinary tract symptoms, comprising administering an effective amount of a compound represented by the formula: 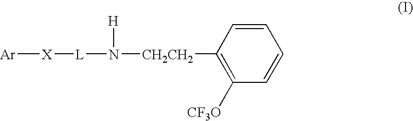
wherein Ar represents a group represented by the formula: 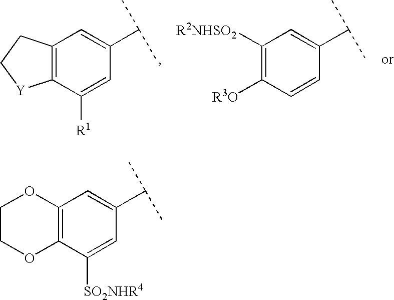
(wherein Y represents methylene or an oxygen atom, R¹ represents aminosulfonyl, C₁₋₆ alkyl-aminosulfonyl, C₁₋₆ alkyl-carbonylamino or C₁₋₆ alkyl-sulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group; and L represents an optionally substituted C₄₋₅ alkylene group, or a salt thereof, or a prodrug thereof to a mammal.

16. Use of a compound represented by the formula: 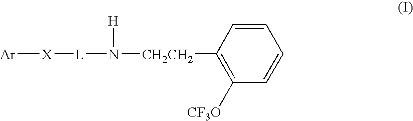
wherein Ar represents a group represented by the formula: 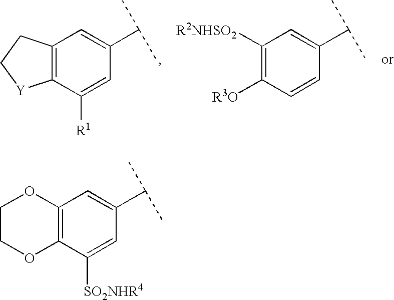
(wherein Y represents methylene or an oxygen atom, R¹ represents aminosulfonyl, C₁₋₆ alkyl-aminosulfonyl, C₁₋₆ alkyl-carbonylamino or C₁₋₆ alkyl-sulfonylamino, R² represents a hydrogen atom or C₁₋₆ alkyl, R³ represents C₁₋₆ alkyl, and R⁴ represents a hydrogen atom or C₁₋₆ alkyl); X represents a carbonyl group, or a methylene group which may be substituted with a hydroxy group; and L represents an optionally substituted C₄₋₅ alkylene group, or a salt thereof, or a prodrug thereof in the preparation of an agent for preventing and/or treating lower urinary tract symptoms.
